(19) United States
(12) Patent Application Publication
(10) Pub. No.: US 2013/0310411 A1
(43) Pub. Date: Nov. 21, 2013

(54) NOVEL ACRIDINE DERIVATIVES

(71) Applicants: Marc Geoffery Hummersone, Nottingham (GB); David Cousin, Nottingham (GB); Mark Frigerio, Nottingham (GB)

(72) Inventors: Marc Geoffery Hummersone, Nottingham (GB); David Cousin, Nottingham (GB); Mark Frigerio, Nottingham (GB)

(73) Assignee: PHARMINOX LIMITED, Nottingham (GB)

(21) Appl. No.: 13/876,656

(22) PCT Filed: Sep. 29, 2011
(86) PCT No.: PCT/GB11/51845

§ 371 Date: Mar. 28, 2013

(30) Foreign Application Priority Data
Sep. 30, 2010 (GB) 1016442.4

(51) Int. Cl. C07D 495/04; C07D 491/052

(52) U.S. Cl. CPC C07D 495/04; C07D 491/052
USPC 514/280; 546/47; 435/184

(57) ABSTRACT

The present invention relates to novel acridine derivatives of formula (I), or pharmaceutically acceptable salts thereof, which are inhibitors of the telomerase enzyme function. These compounds are useful for the treatment cellular proliferation disorders, such as cancer.

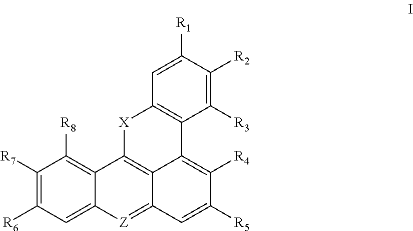

### FIELD OF THE INVENTION

The present invention relates to novel acridine derivatives that function as inhibitors of the telomerase enzyme. More specifically, the present invention relates to novel chromenoacridine and thiochromenoacridine derivatives which are potentially useful therapeutic agents for the treatment and/or prevention of proliferative diseases in which telomerase activity is implicated, such as cancer. The present invention also relates to processes for the preparation of the acridine derivatives described herein, to pharmaceutical compositions comprising them and to their therapeutic uses.

### BACKGROUND OF THE INVENTION

Cancer is caused by uncontrolled and unregulated cellular proliferation. Precisely what causes a cell to become malignant and proliferate in an uncontrolled and unregulated manner has been the focus of intense research over recent decades. This research has led to the identification of numerous tumour-specific or tumour-associated targets, which can then be targeted by anticancer agents.

One particular therapeutic target that has attracted particular attention in the cancer field is the telomerase enzyme, which catalyses the synthesis of telomeres.

Telomeres are nucleoprotein structures at the ends of linear chromosomes which consist of DNA sequences arranged in tandemly repeated units which extend from less than 100 to several thousands of bases. With each cell division cycle, bases are lost from the ends of the chromosomes and, as these telomeres shorten in length, the cell eventually reaches a point at which apoptosis is triggered. Telomerase is a ribonucleoprotein reverse transcriptase that functions to maintain the telomere length above the point at which apoptosis is triggered by utilising its internal RNA template to synthesize telomeric DNA sequences, which compensate for the loss of telomeric DNA (see Blackburn; Annu. Rev. Biochem.; 1992; 61:113-129). This prevents further shortening of the telomeres, and the resulting stabilization of their length contributes to immortalisation.

Telomerase is present in stem and germ line cells of normal tissues, but is absent in most other cells. In addition, telomerase activity, and the associated shortened but stabilized telomeres, have been detected in the majority of tumours examined (and in over 90% of all human cancers examined). As a consequence, the telomerase enzyme is a particularly interesting target for anti-cancer therapy. Furthermore, the absence of telomerase in most normal cells makes this enzyme a particularly attractive target because its inhibition would be expected to cause minimal damage to normal healthy cells and tissues. The fact that tumour cells have shorter telomeres and higher proliferation rates than normal replicative cell populations suggests that a therapeutic telomerase inhibitor may cause tumour cell death well before damage to regenerative tissues occurs, thereby minimising undesirable side effects.

A more detailed discussion of telomeres and telomerase, and their role as anti-proliferative targets is provided in, for example, Sharma et al., Annals of Oncology, 1997, Vol. 8, pp 689-685; Urquidi et al., Ann. Med., 1998, Vol. 30, pp 419-430; Perry et al., Exp. Opin. Ther. Patents, 1998, Vol. 8, No. 12, pp 1567-1586; Autexier, Chemistry & Biology, 1999, Vol. 6, pp R299-R303; and Neidle et al., Anti-Cancer Drug Design, 1999, Vol. 14, pp 341-347.

Certain compounds that target the telomerase enzyme are known. For example, WO 02/30932 describes certain N⁸,N¹³-disubstituted quino[4,3,2-KL]acridium salts that function as inhibitors of telomerase.

However, there still remains a need for alternative and improved telomerase inhibitors. In particular, there remains a need for telomerase inhibitors that possess one more advantageous pharmaceutical properties and which are potentially useful therapeutic agents for the treatment of proliferative disorders such as cancer.

It is therefore an object of the present invention to provide alternative telomerase inhibitors that can be used as pharmaceutical agents for use in the treatment of proliferative disorders such as cancer.

### SUMMARY OF THE INVENTION

In a first aspect, the present invention provides an acridine derivative as defined herein, or a pharmaceutically acceptable salt thereof.

In another aspect, the present invention provides a pharmaceutical composition comprising an acridine derivative, or a pharmaceutically acceptable salt thereof, as defined herein, in admixture with a pharmaceutically acceptable diluent or carrier.

In another aspect, the present invention provides a method of inhibiting telomerase in vitro or in vivo, said method comprising contacting a cell with an effective amount of an acridine derivative, or a pharmaceutically acceptable salt thereof, as defined herein.

In another aspect, the present invention provides a method of inhibiting cell proliferation in vitro or in vivo, said method comprising contacting a cell with an effective amount of an acridine derivative, or a pharmaceutically acceptable salt thereof, as defined herein.

In another aspect, the present invention provides a method of treating a proliferative disorder in a patient in need of such treatment, said method comprising administering to said patient a therapeutically effective amount of an acridine derivative, or a pharmaceutically acceptable salt thereof, or a pharmaceutical composition as defined herein.

In a more particular aspect, the present invention provides a method of treating cancer in a patient in need of such treatment, said method comprising administering to said patient a therapeutically effective amount of an acridine derivative, or a pharmaceutically acceptable salt thereof, or a pharmaceutical composition as defined herein.

In another aspect, the present invention provides an acridine derivative, or a pharmaceutically acceptable salt thereof, or a pharmaceutical composition as defined herein for use in therapy.

In another aspect, the present invention provides an acridine derivative, or a pharmaceutically acceptable salt thereof, or a pharmaceutical composition as defined herein for use in the treatment of a proliferative condition.

In another aspect, the present invention provides an acridine derivative, or a pharmaceutically acceptable salt thereof, or a pharmaceutical composition as defined herein for use in the treatment of cancer. In a particular embodiment, the cancer is a human cancer.

In another aspect, the present invention provides an acridine derivative, or a pharmaceutically acceptable salt thereof, as defined herein for use in the production of a telomerase inhibitory effect.

In another aspect, the present invention provides the use of an acridine derivative, or a pharmaceutically acceptable salt thereof, as defined herein in the manufacture of a medicament for use in the treatment of a proliferative condition.

In another aspect, the present invention provides the use of an acridine derivative, or a pharmaceutically acceptable salt thereof, as defined herein in the manufacture of a medicament for use in the treatment of cancer. Suitably, the medicament is for use in the treatment of human cancers.

In another aspect, the present invention provides the use of an acridine derivative, or a pharmaceutically acceptable salt thereof, as defined herein in the manufacture of a medicament for use in the production of a telomerase inhibitory effect.

The present invention further provides a method of synthesising an acridine derivative, or a pharmaceutically acceptable salt thereof, as defined herein.

In another aspect, the present invention provides an acridine derivative, or a pharmaceutically acceptable salt thereof, obtainable by, or obtained by, or directly obtained by a method of synthesis as defined herein.

In another aspect, the present invention provides novel intermediates as defined herein which are suitable for use in any one of the synthetic methods set out herein.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl. A similar convention applies to other radicals, for example “phenyl(1-6C)alkyl” includes phenyl(1-4C)alkyl, benzyl, 1-phenylethyl and 2-phenylethyl.

The term “(m-nC)” or “(m-nC) group” used alone or as a prefix, refers to any group having m to n carbon atoms.

An “alkylene,” “alkenylene,” or “alkynylene” group is an alkyl, alkenyl, or alkynyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene, ethylene, propylene, 2-methylpropylene, pentylene, and the like.

“(2-6C)alkenylene” means a linear divalent hydrocarbon radical of two to six carbon atoms or a branched divalent hydrocarbon radical of three to six carbon atoms, containing at least one double bond, for example, as in ethenylene, 2,4-pentadienylene, and the like.

“(2-6C)alkynylene” means a linear divalent hydrocarbon radical of two to six carbon atoms or a branched divalent hydrocarbon radical of three to six carbon atoms, containing at least one triple bond, for example, as in ethynylene, propynylene, and butynylene and the like.

“(3-8C)cycloalkyl” means a hydrocarbon ring containing from 3 to 8 carbon atoms, for example, cyclopropyl, cyclobutyl, cyclopentyl, cyclohexyl, cycloheptyl or bicyclo[2.2.1]heptyl.

“(3-8C)cycloalkenyl” means a hydrocarbon ring containing at least one double bond, for example, cyclobutenyl, cyclopentenyl, cyclohexenyl or cycloheptenyl, such as 3-cyclohexen-1-yl, or cyclooctenyl.

“(3-8C)cycloalkyl-(1-6C)alkylene” means a (3-8C)cycloalkyl group covalently attached to a (1-6C)alkylene group, both of which are defined herein.

The term “halo” refers to fluoro, chloro, bromo and iodo.

The term “heterocyclyl”, “heterocyclic” or “heterocycle” means a non-aromatic saturated or partially saturated monocyclic, fused, bridged, or spiro bicyclic heterocyclic ring system(s). The term heterocyclyl includes both monovalent species and divalent species. Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocycles contain from about 7 to about 17 ring atoms, suitably from 7 to 12 ring atoms. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydro-oxathiolyl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane and quinuclidine.

“Heterocyclyl(1-6C)alkyl” means a heterocyclyl group covalently attached to a (1-6C)alkylene group, both of which are defined herein.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:
a) a benzene ring fused to a 5- or 6-membered ring containing 1, 2 or 3 ring heteroatoms;
b) a pyridine ring fused to a 5- or 6-membered ring containing 1, 2 or 3 ring heteroatoms;
c) a pyrimidine ring fused to a 5- or 6-membered ring containing 1 or 2 ring heteroatoms;
d) a pyrrole ring fused to a 5- or 6-membered ring containing 1, 2 or 3 ring heteroatoms;
e) a pyrazole ring fused to a 5- or 6-membered ring containing 1 or 2 ring heteroatoms;
f) a pyrazine ring fused to a 5- or 6-membered ring containing 1 or 2 ring heteroatoms;
g) an imidazole ring fused to a 5- or 6-membered ring containing 1 or 2 ring heteroatoms;
h) an oxazole ring fused to a 5- or 6-membered ring containing 1 or 2 ring heteroatoms;
i) an isoxazole ring fused to a 5- or 6-membered ring containing 1 or 2 ring heteroatoms;
j) a thiazole ring fused to a 5- or 6-membered ring containing 1 or 2 ring heteroatoms;
k) an isothiazole ring fused to a 5- or 6-membered ring containing 1 or 2 ring heteroatoms;
l) a thiophene ring fused to a 5- or 6-membered ring containing 1, 2 or 3 ring heteroatoms;
m) a furan ring fused to a 5- or 6-membered ring containing 1, 2 or 3 ring heteroatoms;
n) a cyclohexyl ring fused to a 5- or 6-membered heteroaromatic ring containing 1, 2 or 3 ring heteroatoms; and
o) a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

“Heteroaryl(1-6C)alkyl” means a heteroaryl group covalently attached to a (1-6C)alkylene group, both of which are defined herein. Examples of heteroaralkyl groups include pyridin-3-ylmethyl, 3-(benzofuran-2-yl)propyl, and the like.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In particular embodiment, an aryl is phenyl.

The term “aryl(1-6C)alkyl” means an aryl group covalently attached to a (1-6C)alkylene group, both of which are defined herein. Examples of aryl-(1-6C)alkyl groups include benzyl, phenylethyl, and the like

This specification also makes use of several composite terms to describe groups comprising more than one functionality. Such terms will be understood by a person skilled in the art. For example heterocyclyl(m-nC)alkyl comprises (m-nC)alkyl substituted by heterocyclyl.

The term “optionally substituted” refers to either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups.

The phrase “compound of the invention” means those compounds which are disclosed herein, both generically and specifically.

### Acridine Derivatives

According to a first aspect of the present invention there is provided an acridine derivative of formula I:

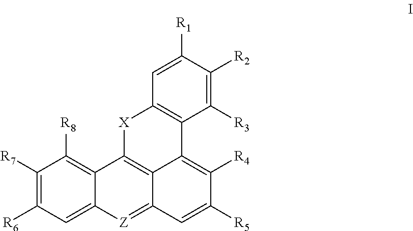

wherein:

X is O, S, SO or SO₂,

Z is N or N⁺-Q, wherein Q is selected from O⁻, (1-6C)alkyl, (2-6C)alkenyl or (2-6C)alkynyl, or Q is a group of the formula:

-L¹-Q¹

wherein:
L¹ is (1-6C)alkylene, (2-6C)alkenylene or (2-6C)alkynylene, each of which is optionally substituted with one or more (1-4C)alkyl groups;
Q¹ is selected from −OR₉, —NR₉R₁₀, —S(O)ₚR₉ (wherein p is 0, 1 or 2), —C(O)R₉, —C(O)OR₉, —OC(O)R₉, —C(O)NR₉R₁₀, —N(R₁₀)C(O)R₉, —N(R₁₀)CON(R₁₀)R₉—, —SO₂N(R₉)—, —N(R₉)SO₂—, (3-8C)cycloalkyl, aryl, heterocyclyl, heteroaryl, and wherein a (3-8C)cycloalkyl, aryl, heterocyclyl, heteroaryl group is optionally substituted by one or more substituents independently selected from halogeno, cyano, nitro, hydroxy, amino or (1-4C)alkoxy;
R₉ is selected from hydrogen, (1-6C)alkyl, (3-8C)cycloalkyl, aryl, heterocyclyl, heteroaryl, and wherein a (1-6C)alkyl, (3-8C)cycloalkyl, aryl, heterocyclyl, heteroaryl group is optionally substituted by one or more substituents independently selected from halogeno, cyano, nitro, hydroxy, amino or (1-4C)alkoxy;
R₁₀ is selected from hydrogen of (1-6C)alkyl;

R₁, R₂, R₃, R₄, R₅, R₆, R₇ and R₈ are each independently selected from hydrogen, halogeno, trifluoromethyl, cyano, nitro, hydroxy, mercapto, amino, formyl, carboxy, carbamoyl, ureido, (1-6C)alkyl, (2-6C)alkenyl, (2-6C)alkynyl,

or a group of the formula:

-L²-L³-R₂₀

wherein
L² is absent or a linker group of the formula —[CR₁₁R₁₂]ₙ— in which n is an integer selected from 1, 2, 3 or 4 and R₁₁ and R₁₂ are each independently selected from hydrogen or (1-4C)alkyl;
L³ is absent or is selected from O, S, SO, SO₂, N(R₁₃), C(O), CH(OR₁₃), C(O)O, OC(O), C(O)N(R₁₃), N(R₁₃)C(O), N(R₁₃)C(O)N(R₁₄), S(O)₂N(R₁₃), or N(R₁₃)SO₂, wherein R₁₃ and R₁₄ are each independently selected from hydrogen or (1-4C)alkyl; and
   R₂₀ is (1-6C)alkyl, aryl, aryl-(1-6C)alkyl, (3-6C)cycloalkyl, (3-6C)cycloalkyl-(1-6C)alkyl, (3-6C)cycloalkenyl, (3-6C)cycloalkenyl-(1-6C)alkyl, heteroaryl, heteroaryl-(1-6C)alkyl, heterocyclyl or heterocyclyl-(1-6C)alkyl and wherein R₂₀ is optionally further substituted by one or more substituents independently selected from halogeno, cyano, nitro, hydroxy, amino or (1-4C)alkoxy;
      or a pharmaceutically acceptable salt thereof.

Particular novel compounds of the invention include, for example, compounds of the formula I, or pharmaceutically acceptable salts thereof, wherein, unless otherwise stated, each of X, Z, Q, R₁, R₂, R₃, R₄, R₅, R₆, R₇, and R₈ has any of the meanings defined hereinbefore or in any of paragraphs (1) to (16) hereinafter:—
(1) X is O or S;
(2) X is O;
(3) X is S;
(4) Z is N or N⁺-Q, wherein Q is as defined in any one of paragraphs (7) to (12) below;
(5) Z is N;
(6) Z is N⁺-Q;
(7) Q is selected from O⁻, (1-6C)alkyl, (2-6C)alkenyl or (2-6C)alkynyl, or Q is a group of the formula:

-L¹-Q¹
wherein:
L¹ is (1-6C)alkylene which is optionally substituted with one or more (1-4C)alkyl groups;

Q¹ is selected from —OR₉, —NR₉R₁₀, —S(O)ₚR₉ (wherein p is 0, 1 or 2), —C(O)R₉, —C(O)OR₉, —OC(O)R₉, —C(O)NR₉R₁₀, —N(R₁₀)C(O)R₉, —N(R₁₀)CON(R₁₀)R₉—, —SO₂N(R₉)—, —N(R₉)SO₂—, (3-8C)cycloalkyl, aryl, heterocyclyl, heteroaryl, and wherein a (3-8C)cycloalkyl, aryl, heterocyclyl or heteroaryl group is optionally substituted by one or more substituents independently selected from halogeno, cyano, nitro, hydroxy, amino or (1-4C)alkoxy;
R₉ is selected from hydrogen, (1-6C)alkyl, (3-8C)cycloalkyl, aryl, heterocyclyl, heteroaryl, and wherein a (1-6C)alkyl, (3-8C)cycloalkyl, aryl, heterocyclyl, heteroaryl group is optionally substituted by one or more substituents independently selected from halogeno, cyano, nitro, hydroxy, amino or (1-4C)alkoxy;
R₁₀ is selected from hydrogen of (1-6C)alkyl;
(8) Q is selected from O⁻, (1-6C)alkyl, (2-6C)alkenyl or (2-6C)alkynyl, or Q is a group of the formula:

-L¹-Q¹
wherein:
L¹ is (1-4C)alkylene which is optionally substituted with one or more (1-2C)alkyl groups;
Q¹ is selected from −OR₉, —NR₉R₁₀, —S(O)ₚR₉ (wherein p is 0, 1 or 2), —C(O)R₉, —C(O)OR₉, —OC(O)R₉, —C(O)NR₉R₁₀, —N(R₁₀)C(O)R₉, —N(R₁₀)CON(R₁₀)R₉—, —SO₂N(R₉)—, —N(R₉)SO₂—, (3-6C)cycloalkyl, aryl, heterocyclyl, heteroaryl, and wherein a (3-6C)cycloalkyl, aryl, heterocyclyl or heteroaryl group is optionally substituted by one or more substituents independently selected from halogeno, cyano, nitro, hydroxy, amino or (1-4C)alkoxy;
R₉ is selected from hydrogen, (1-4C)alkyl, (3-6C)cycloalkyl, aryl, heterocyclyl, heteroaryl, and wherein a (1-4C)alkyl, (3-6C)cycloalkyl, aryl, heterocyclyl, heteroaryl group is optionally substituted by one or more substituents independently selected from halogeno, cyano, nitro, hydroxy, amino or (1-4C)alkoxy;
R₁₀ is selected from hydrogen of (1-2C)alkyl;
(9) Q is selected from O⁻ or (1-6C)alkyl or Q is a group of the formula:

-L¹-Q¹
wherein:
L¹ is (1-2C)alkylene;
Q¹ is selected from −OR₉, —NR₉R₁₀, —S(O)ₚR₉ (wherein p is 0, 1 or 2), —C(O)R₉, —C(O)OR₉, —OC(O)R₉, —C(O)NR₉R₁₀, —N(R₁₀)C(O)R₉, —N(R₁₀)CON(R₁₀)R₉—, —SO₂N(R₉)—, —N(R₉)SO₂—, (3-6C)cycloalkyl, aryl, heterocyclyl, heteroaryl, and wherein a (3-6C)cycloalkyl, aryl, heterocyclyl or heteroaryl group is optionally substituted by one or more substituents independently selected from halogeno, cyano, nitro, hydroxy, amino or (1-4C)alkoxy;
R₉ is selected from hydrogen or (1-4C)alkyl;
R₁₀ is selected from hydrogen of (1-2C)alkyl;
(10) Q is selected from O⁻, (1-6C)alkyl or Q is a group of the formula:

-L¹-Q¹
wherein L¹ and Q¹ have any one of the definitions set out herein
(11) Q is selected from O⁻ or (1-2C)alkyl;
(12) Q is selected from O⁻ or methyl;
(13) R₁, R₂, R₃, R₄, R₅, R₆, R₇ and R₈ are each independently selected from hydrogen, halogeno, trifluoromethyl, cyano, nitro, hydroxy, mercapto, amino, formyl, carboxy, carbamoyl, ureido, (1-6C)alkyl, (2-6C)alkenyl, (2-6C)alkynyl, or a group of the formula:

-L²-L³-R₂₀
wherein
   L² is absent or a linker group of the formula —[CR₁₁R₁₂]ₙ— in which n is an integer selected from 1 or 2, and R₁₁ and R₁₂ are each independently selected from hydrogen or (1-2C)alkyl;
   L³ is absent or is selected from O, S, SO, SO₂, N(R₁₃), C(O), CH(OR₁₃), C(O)O, OC(O), C(O)N(R₁₃), N(R₁₃)C(O), N(R₁₃)C(O)N(R₁₄), S(O)₂N(R₁₃), or N(R₁₃)SO₂, wherein R₁₃ and R₁₄ are each independently selected from hydrogen or (1-2C)alkyl; and
   R₂₀ is (1-6C)alkyl, aryl, aryl-(1-6C)alkyl, (3-6C)cycloalkyl, (3-6C)cycloalkyl-(1-6C)alkyl, heteroaryl, heteroaryl-(1-6C)alkyl, heterocyclyl or heterocyclyl-(1-6C)alkyl and wherein R₂₀ is optionally further substituted by one or more substituents independently selected from halogeno, cyano, nitro, hydroxy, amino or (1-4C)alkoxy;
(14) R₁, R₂, R₃, R₄, R₅, R₆, R₇ and R₈ are each independently selected from hydrogen, halogeno, trifluoromethyl, cyano, nitro, hydroxy, mercapto, amino, carboxy, carbamoyl, ureido, (1-6C)alkyl, (2-6C)alkenyl, (2-6C)alkynyl, or a group of the formula:

-L²-L³-R₂₀
wherein
   L² is absent or a linker group of the formula —[CR₁₁R₁₂]ₙ— in which n is an integer selected from 1 or 2, and R₁₁ and R₁₂ are each independently selected from hydrogen or (1-2C)alkyl;
   L³ is absent or is selected from O, S, SO, SO₂, N(R₁₃), C(O), CH(OR₁₃), C(O)O, OC(O), C(O)N(R₁₃), N(R₁₃)C(O), S(O)₂N(R₁₃), or N(R₁₃)SO₂, wherein R₁₃ and R₁₄ are each independently selected from hydrogen or (1-2C)alkyl; and
   R₂₀ is (1-6C)alkyl, aryl, aryl-(1-6C)alkyl, (3-6C)cycloalkyl, (3-6C)cycloalkyl-(1-6C)alkyl, heteroaryl, heteroaryl-(1-6C)alkyl, heterocyclyl or heterocyclyl-(1-6C)alkyl and wherein R₂₀ is optionally further substituted by one or more substituents independently selected from halogeno, cyano, nitro, hydroxy, amino or (1-4C)alkoxy;
(15) R₁, R₂, R₃, R₄, R₅, R₆, R₇ and R₈ are each independently selected from hydrogen, halogeno, trifluoromethyl, cyano, nitro, hydroxy, mercapto, amino, carbamoyl, (1-6C)alkyl, or a group of the formula:

-L²-L³-R₂₀
wherein
   L² is absent or a methylene linker;
   L³ is absent or is selected from O, S, SO, SO₂, N(R₁₃), C(O), CH(OR₁₃), C(O)O, OC(O), C(O)N(R₁₃), N(R₁₃)C(O), N(R₁₃)C(O)N(R₁₄), S(O)₂N(R₁₃), or N(R₁₃)SO₂, wherein R₁₃ and R₁₄ are each independently selected from hydrogen or (1-2C)alkyl; and
   R₂₀ is (1-6C)alkyl or (3-6C)cycloalkyl, and wherein R₂₀ is optionally further substituted by one or more substituents independently selected from halogeno, cyano, nitro, hydroxy, amino or (1-2C)alkoxy;
(16) R₁, R₂, R₃, R₄, R₅, R₆, R₇ and R₈ are each independently selected from hydrogen, halogeno, trifluoromethyl, cyano, nitro, hydroxy, mercapto, amino, carbamoyl, (1-4C)alkyl, or a group of the formula:

-L²-L³-R₂₀
wherein
   L² is absent;
   L³ is absent or is selected from O, S, SO, SO₂, N(R₁₃), C(O), CH(OR₁₃), C(O)O, OC(O), C(O)N(R₁₃), N(R₁₃)C(O), S(O)₂N(R₁₃), or N(R₁₃)SO₂, wherein R₁₃ and R₁₄ are each independently selected from hydrogen or (1-2C)alkyl; and
   R₂₀ is (1-4C)alkyl which is optionally further substituted by one or more substituents independently selected from halogeno, cyano, nitro, hydroxy, amino or (1-2C)alkoxy.

In a particular group of compounds of the invention, R₁, R₂, R₃, R₄, R₅, R₆, R₇ and R₈ each have any one of the definitions set out herein, with the proviso that only up to four of R₁, R₂, R₃, R₄, R₅, R₆, R₇ and R₈ are a substituent group other than hydrogen.

In a particular group of compounds of the invention, R₁, R₂, R₃, R₄, R₅, R₆, R₇ and R₈ each have any one of the definitions set out herein, with the proviso that only up to three of R₁, R₂, R₃, R₄, R₅, R₆, R₇ and R₈ are a substituent group other than hydrogen.

In a particular group of compounds of the invention, R₁, R₂, R₃, R₄, R₅, R₆, R₇ and R₈ each have any one of the definitions set out herein, with the proviso that only one or two of R₁, R₂, R₃, R₄, R₅, R₆, R₇ and R₈ is/are a substituent group other than hydrogen.

In a particular group of compounds of the invention, R₁, R₂, R₃, R₄, R₅, R₆, R₇ and R₈ each have any one of the definitions set out herein, with the proviso that only one of R₁, R₂, R₃,

R₄, R₅, R₆, R₇ and R₈ is a substituent group other than hydrogen.

In a particular group of compounds of the invention, R₈ is hydrogen, i.e. the compounds have the structural formula II shown below:

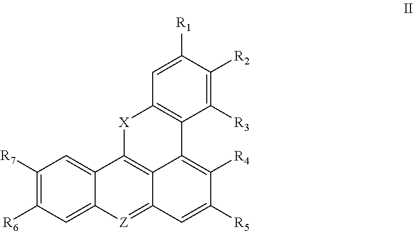

wherein X, Z, R₁, R₂, R₃, R₄, R₅, R₆ and R₇ have any one of the meanings defined herein, or a pharmaceutically acceptable salt thereof.

In a further group of compounds of the invention, the groups R₄, R₅, R₆, R₇ and R₈ are all hydrogen, i.e. the compounds have the structural formula III shown below:

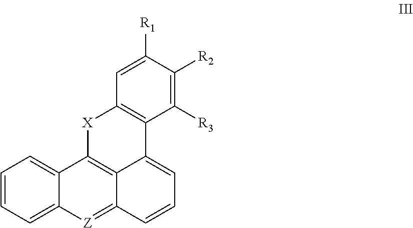

wherein X, Z, R₁, R₂ and R₃ have any one of the meanings defined herein, or a pharmaceutically acceptable salt thereof.

In yet a further group of compounds of the invention, the groups R₃, R₄, R₅, R₆, R₇ and R⁸ are all hydrogen, i.e. the compounds have the structural formula IV shown below:

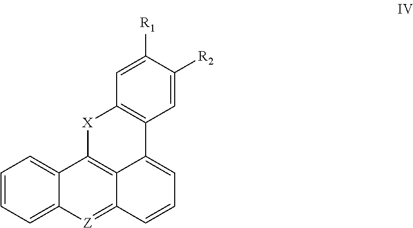

wherein X, Z, R₁ and R₂ have any one of the meanings defined herein; or a pharmaceutically acceptable salt thereof.

In a particular group of compounds of formula IV, one of R₁ or R₂ is hydrogen.

The various functional groups and substituents making up the compounds of the formula I are typically chosen such that the molecular weight of the compound of the formula I does not exceed 1000. More usually, the molecular weight of the compound will be less than 750, for example less than 700, or less than 650, or less than 600, or less than 550. More preferably, the molecular weight is less than 525 and, for example, is 500 or less.

Particular compounds of the invention include any one of the following:
chromeno[4,3,2-kl]acridine;
thiochromeno[4,3,2-kl]acridine;
chromeno[4,3,2-kl]acridine 8-oxide;
N-(chromeno[4,3,2-kl]acridin-3-yl)acetamide;
8-methylchromeno[4,3,2-kl]acridin-8-ium;
8-methylthiochromeno[4,3,2-kl]acridin-8-ium;
8-(2-ethoxy-2-oxoethyl)chromeno[4,3,2-kl]acridin-8-ium;
acetamido-8-methylchromeno[4,3,2-kl]acridin-8-ium;
3-methoxy-8-methylthiochromeno[4,3,2-kl]acridin-8-ium;
4-methoxy-8-methylthiochromeno[4,3,2-kl]acridin-8-ium;
3,10-dimethoxy-8-methylthiochromeno[4,3,2-kl]acridin-8-ium;
10-methoxy-2-(methoxycarbonyl)-8-methylthiochromeno[4,3,2-kl]acridin-8-ium;
10-methoxy-4-(methoxycarbonyl)-8-methylthiochromeno[4,3,2-kl]acridin-8-ium;
3-acetoxy-8-methylthiochromeno[4,3,2-kl]acridin-8-ium;
3-hydroxy-8-methylthiochromeno[4,3,2-kl]acridin-8-ium;
2-(methoxycarbonyl)-8-methylthiochromeno[4,3,2-kl]acridin-8-ium;
4-(methoxycarbonyl)-8-methylthiochromeno[4,3,2-kl]acridin-8-ium;
3-(methoxymethoxy)thiochromeno[4,3,2-kl]acridine;
4-acetamido-8-methylthiochromeno[4,3,2-kl]acridin-8-ium;
5,10-dimethoxy-8-methylthiochromeno[4,3,2-kl]acridin-8-ium;
3-(methoxycarbonyl)-8-methylthiochromeno[4,3,2-kl]acridin-8-ium
   or a pharmaceutically acceptable salt thereof.

Further specific compounds of the invention include any one of the following:
chromeno[4,3,2-kl]acridine;
thiochromeno[4,3,2-kl]acridine;
chromeno[4,3,2-kl]acridine 8-oxide;
N-(chromeno[4,3,2-kl]acridin-3-yl)acetamide;
8-methylchromeno[4,3,2-kl]acridin-8-ium chloride;
8-methylthiochromeno[4,3,2-kl]acridin-8-ium chloride;
8-(2-ethoxy-2-oxoethyl)chromeno[4,3,2-kl]acridin-8-ium iodide;
acetamido-8-methylchromeno[4,3,2-kl]acridin-8-ium chloride;
3-methoxy-8-methylthiochromeno[4,3,2-kl]acridin-8-ium chloride;
4-methoxy-8-methylthiochromeno[4,3,2-kl]acridin-8-ium iodide;
3,10-dimethoxy-8-methylthiochromeno[4,3,2-kl]acridin-8-ium chloride;
10-methoxy-2-(methoxycarbonyl)-8-methylthiochromeno[4,3,2-kl]acridin-8-ium chloride;
10-methoxy-4-(methoxycarbonyl)-8-methylthiochromeno[4,3,2-kl]acridin-8-ium chloride;
3-acetoxy-8-methylthiochromeno[4,3,2-kl]acridin-8-ium chloride;
3-hydroxy-8-methylthiochromeno[4,3,2-kl]acridin-8-ium chloride;
2-(methoxycarbonyl)-8-methylthiochromeno[4,3,2-kl]acridin-8-ium chloride;
4-(methoxycarbonyl)-8-methylthiochromeno[4,3,2-kl]acridin-8-ium iodide;
3-(methoxymethoxy)thiochromeno[4,3,2-kl]acridine;
4-acetamido-8-methylthiochromeno[4,3,2-kl]acridin-8-ium iodide;
5,10-dimethoxy-8-methylthiochromeno[4,3,2-kl]acridin-8-ium iodide;
3-(methoxycarbonyl)-8-methylthiochromeno[4,3,2-kl]acridin-8-ium iodide and chloride.

In an embodiment of the invention, the compounds of the invention are as defined herein with the proviso that the compound is not one or more of the specific compounds listed above.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Compounds of formula I in which Z is N⁺-Q in particular are cationic and will be associated with one or more counter anions. The compound of formula I possesses a +1 charge. The anion may carry a −1 charge, in which case the molar ratio of anion:cation is 1:1, or, alternatively, the anion may carry a −2 or −3 charge, in which case the molar ratio of anion:cation is will be 1:2 or 1:3, respectively.

In one embodiment, the anion is independently derived from one or more of the following acids: hydrochloric, hydrobromic, hydroiodic, sulfuric, sulfurous, nitric, nitrous, phosphoric, phosphorous acetic, propionic, succinic, gycolic, stearic, lactic, malic, tartaric, citric, ascorbic, maleic, hydroxymaleic, phenylacetic, glutamic, benzoic, salicylic, sulfanilic, 2-acetyoxybenzoic, fumaric, toluenesulfonic, methanesulfonic, ethanesulfonic, ethane disulfonic, oxalic, isethionic, and valeric.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable minor images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess telomerase inhibitory activity.

The present invention also encompasses compounds of the invention as defined herein which comprise one or more isotopic substitutions. For example, H may be in any isotopic form, including ¹H, ²H(D), and ³H (T); C may be in any isotopic form, including ¹²C, ¹³C, and ¹⁴C; and O may be in any isotopic form, including ¹⁶0 and ¹⁸0; and the like.

It is also to be understood that certain compounds of the formula I may exist in solvated as well as unsolvated forms such as, for example, hydrated forms. It is to be understood that the invention encompasses all such solvated forms that possess telomerase inhibitory activity.

It is also to be understood that certain compounds of the formula I may exhibit polymorphism, and that the invention encompasses all such forms that possess telomerase inhibitory activity.

Compounds of the formula I may exist in a number of different tautomeric forms and references to compounds of the formula I include all such forms. For the avoidance of doubt, where a compound can exist in one of several tautomeric forms, and only one is specifically described or shown, all others are nevertheless embraced by formula I. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

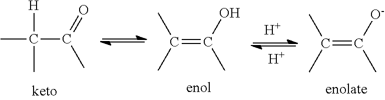

Compounds of the formula I containing an amine function may also form N-oxides. A reference herein to a compound of the formula I that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

The compounds of formula I may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the formula I and in-vivo cleavable amide derivatives that may be formed at a carboxy group or an amino group in a compound of the formula I.

Accordingly, the present invention includes those compounds of the formula I as defined hereinbefore when made available by organic synthesis and when made available within the human or animal body by way of cleavage of a pro-drug thereof. Accordingly, the present invention includes those compounds of the formula I that are produced by organic synthetic means and also such compounds that are produced in the human or animal body by way of metabolism of a precursor compound, that is a compound of the formula I may be a synthetically-produced compound or a metabolically-produced compound.

A suitable pharmaceutically acceptable pro-drug of a compound of the formula I is one that is based on reasonable medical judgement as being suitable for administration to the human or animal body without undesirable pharmacological activities and without undue toxicity.

Various forms of pro-drug have been described, for example in the following documents:—

a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);

### d) H. Bundgaard, Advanced Drug Delivery Reviews, 8, 1-38 (1992);

### e) H. Bundgaard, et al., Journal of Pharmaceutical Sciences, 77, 285 (1988);

f) N. Kakeya, et al., Chem. Pharm. Bull., 32, 692 (1984);

### g) T. Higuchi and V. Stella, “Pro-Drugs as Novel Delivery Systems”, A.C.S. Symposium Series, Volume 14; and

h) E. Roche (editor), “Bioreversible Carriers in Drug Design”, Pergamon Press, 1987.

A suitable pharmaceutically acceptable pro-drug of a compound of the formula I that possesses a carboxy group is, for example, an in vivo cleavable ester thereof. An in vivo cleavable ester of a compound of the formula I containing a carboxy group is, for example, a pharmaceutically acceptable ester which is cleaved in the human or animal body to produce the parent acid. Suitable pharmaceutically acceptable esters for carboxy include C₁₋₆alkyl esters such as methyl, ethyl and tert-butyl, C₁₋₆alkoxymethyl esters such as methoxymethyl esters, C₁₋₆alkanoyloxymethyl esters such as pivaloyloxymethyl esters, 3-phthalidyl esters, C₃₋₈cycloalkylcarbonyloxy-C₁₋₆alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C₁₋₆alkoxycarbonyloxy-C₁₋₆alkyl esters such as methoxycarbonyloxymethyl and 1-methoxycarbonyloxyethyl esters.

A suitable pharmaceutically acceptable pro-drug of a compound of the formula I that possesses a hydroxy group is, for example, an in vivo cleavable ester or ether thereof. An in vivo cleavable ester or ether of a compound of the formula I containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁₋₁₀alkanoyl groups such as acetyl, benzoyl, phenylacetyl and substituted benzoyl and phenylacetyl groups, C₁₋₁₀alkoxycarbonyl groups such as ethoxycarbonyl, N,N—(C₁₋₆)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable pro-drug of a compound of the formula I that possesses a carboxy group is, for example, an in vivo cleavable amide thereof, for example an amide formed with an amine such as ammonia, a C₁₋₄alkylamine such as methylamine, a (C₁₋₄alkyl)₂amine such as dimethylamine, N-ethyl-N-methylamine or diethylamine, a C₁₋₄alkoxy-C₂₋₄alkylamine such as 2-methoxyethylamine, a phenyl-C₁₋₄alkylamine such as benzylamine and amino acids such as glycine or an ester thereof.

A suitable pharmaceutically acceptable pro-drug of a compound of the formula I that possesses an amino group is, for example, an in vivo cleavable amide derivative thereof. Suitable pharmaceutically acceptable amides from an amino group include, for example an amide formed with C₁₋₁₀alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl.

The in vivo effects of a compound of the formula I may be exerted in part by one or more metabolites that are formed within the human or animal body after administration of a compound of the formula I. As stated hereinbefore, the in vivo effects of a compound of the formula I may also be exerted by way of metabolism of a precursor compound (a pro-drug).

It shall also be appreciated that compounds of formula I may also be covalently linked (at any suitable position) to other groups such as, for example, solubilising moieties (for example, PEG polymers), moieties that enable them to be bound to a solid support (such as, for example, biotin-containing moieties), and targeting ligands (such as antibodies or antibody fragments).

### Synthesis

The compounds of the present invention can be prepared in accordance with the general process described below and by the processes described in the accompanying examples.

In the description of the synthetic methods described below and in the referenced synthetic methods that are used to prepare the staring materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined below, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

In a particular aspect, the present invention provides a process for the production of a compound of the formula I, or a pharmaceutically acceptable salt thereof, which comprises:

the intra-molecular free radical or palladium mediated cyclisation of a compound of formula A

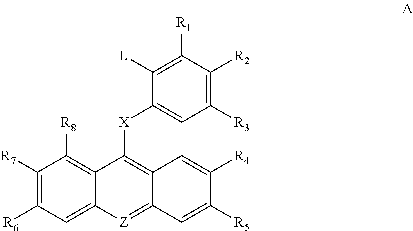

wherein X, R₁, R₂, R₃, R₄, R₅, R₆, R₇ and R₈ each have any one of the meanings defined hereinbefore, Z is N, and L is halogen atom or other appropriate leaving group (e.g. trifluoromethanesulfonate);

and optionally thereafter, and if necessary:

(i) removing any protecting groups present;

(ii) converting the compound formula I into another compound of formula I; and/or

(iii) forming a pharmaceutically acceptable salt thereof.

In a particular aspect, the present invention provides a process for the production of a compound of the formula I, or a pharmaceutically acceptable salt thereof, which comprises:

the intra-molecular free radical or palladium mediated cyclisation of a compound of formula B

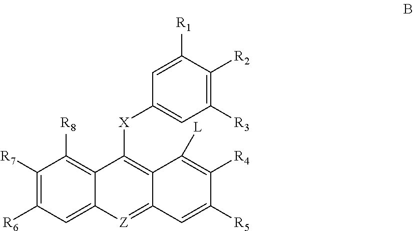

wherein X, R₁, R₂, R₃, R₄, R₅, R₆, R₇ and R₈ each have any one of the meanings defined hereinbefore, Z is N, and L is halogen atom or other appropriate leaving group (e.g. trifluoromethanesulfonate);

and optionally thereafter, and if necessary:

(i) removing any protecting groups present;

(ii) converting the compound formula I into another compound of formula I; and/or

(iii) forming a pharmaceutically acceptable salt thereof.

Suitably L is fluoro, chloro or bromo, particularly chloro or bromo, or another suitable leaving group such as trifluoromethanesulfonate.

The intra-molecular free radical cyclisation of a compound of formula A or B is carried out using techniques generally known in the art (see, for example, Antitumour polycyclic acridines. Part 10.1 Synthesis of penta- and hexa-cyclic heteroaromatic systems by radical cyclisations of substituted 9-anilinoacridines: J. Chem. Soc., Perkin Trans. 1, 2001, 3180-3185).

Suitably, the intra-molecular free radical cyclisation of a compound of formula A or B is carried out in the presence of a suitable free radical initiator, such as azobisisobutyronitrile (AIBN), 2,2′-azobis-2-methylpropionitrile, benzoyl peroxide or 1,1′-azobis-1-cyclohexanenitrile, and a radical reducing agent such as tributylstannane, trimethylstannane, triphenylstannane or tris(trimethylsilyl)silane or tris(tributyl)silane.

In an embodiment, the reaction is carried out using azobisisobutyronitrile (AIBN) and tributylstannane.

The intra-molecular free radical cyclisation reaction is carried out in the presence of a suitable solvent. Any suitable solvent or solvent mixture may be used for this reaction. A person skilled in the art will know how to select suitable solvents or solvent mixtures for use in these reactions. Particular examples of suitable solvents include anhydrous toluene, benzene, nitrobenzezne, xylene, or mixtures thereof.

Suitably, the reaction is carried out in anhydrous conditions and in the presence of an inert atmosphere, such as argon or nitrogen.

The reaction is carried out an elevated temperature, suitably within the range of 60 to 160° C. or, more suitably 100 to 140° C., for a suitable time period of, for example, 12 hours to 7 days. A person skilled in the art will be able to select appropriate reaction conditions to use in order to facilitate this reaction.

The resultant compound of formula I can be isolated and purified using techniques well known in the art.

As stated above, the compound of formula I may, if necessary, be further reacted to:

(i) remove any protecting groups present;

(ii) convert the compound formula I into another compound of formula I; and/or

(iii) forming a pharmaceutically acceptable salt thereof.

An example of (ii) above is when compounds of formula I in which Z is N are produced by the process defined above and are then subsequently converted into a compound of formula I in which Z is N⁺-Q by reacting the compound in which Z is N with a compound of the formula:

Q-P

wherein Q is as defined hereinbefore and P is a suitable leaving group, such as halo or trifluoromethanesulfonate or Meerwein salt or dimethyl sulfate.

Alternatively, one or more of the groups R₁ to R₈ may be reacted to change the nature of the group and provide an alternative compound of formula I.

The process defined herein may further comprise the step of subjecting the compound of formula Ito a salt exchange, particularly in situations where the compound of formula I is formed as a mixture of different salt forms. The salt exchange suitably comprises immobilising the compound of formula I on a suitable solid support or resin, and eluting the compounds with an appropriate acid to yield a single salt of the compound of formula I.

The compounds of formula A or B can be prepared by processes known in the art.

In a particular embodiment, the compound of formula A is prepared by reacting a compound of formula C:

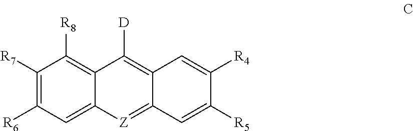

wherein D is halo and Z, R₄, R₅, R₆, R₇ and R₈ are as hereinbefore defined;
with a compound of formula D

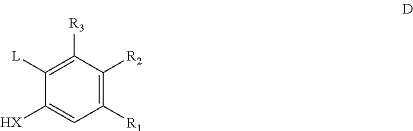

wherein L, X, R₁, R₂ and R₃ are as hereinbefore defined.

Compounds of formula C above can be prepared by techniques known in the art, such as, for example, using the process described in Bioorg. Med. Chem. 2006, 4, 334.

This reaction may be carried out in any suitable solvent. A person skilled in the art will know how to select suitable solvents or solvent mixtures for use in this reaction. Particular examples of suitable solvents include anhydrous dimethylformamide, THF, DMSO or mixtures thereof.

Suitably, the reaction is carried out in anhydrous conditions and in the presence of an inert atmosphere, such as argon or nitrogen.

The reaction may be carried out at room temperature or at an elevated temperature, for example within the range of 100 to 160° C., more preferably 120 to 150° C., for a suitable time period, for example between 2 and 24 hours. A person skilled in the art will be able to select appropriate reaction conditions to use to facilitate this reaction.

The resultant compound of formula A can be isolated and purified using techniques well known in the art.

Suitably the group D is fluoro, chloro or bromo, especially chloro.

In a further embodiment, the compound of formula B is prepared by reacting a compound of formula E:

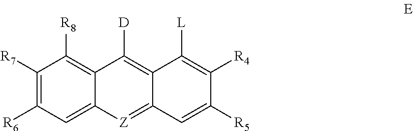

wherein D, L, Z, R₄, R₅, R₆, R₇ and R₈ are as hereinbefore defined;
with a compound of formula F

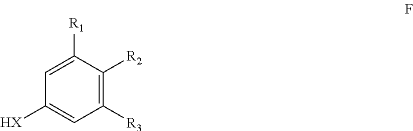

wherein X, R₁, R₂ and R₃ are as hereinbefore defined.

In this reaction, D is suitably chloro and L is suitably bromo.

Compounds of formula E above can be prepared by techniques known in the art, such as, for example, by the adaptation of the process described in Bioorg. Med. Chem. 2006, 4, 334.

These reactions may be carried out in any suitable solvent. A person skilled in the art will know how to select suitable solvents or solvent mixtures for use in these reactions. Particular examples of suitable solvents include anhydrous dimethylformamide, THF, DMSO or mixtures thereof.

Suitably, the reaction is carried out in anhydrous conditions and in the presence of an inert atmosphere, such as argon or nitrogen.

Suitably, the reaction is carried out in the presence of a suitable reducing agent, such as sodium hydride.

The reaction may be carried out at room temperature or, more preferably, under cooled conditions, e.g. a temperature of 0-5° C., for a suitable time period, for example between 2 and 24 hours. A person skilled in the art will be able to select appropriate reaction conditions to use to facilitate this reaction.

The resultant compound of formula B can be isolated and purified using techniques well known in the art.

In a further aspect of the invention, there is provided a compound of formula I obtainable by a process as defined herein.

In a further aspect of the invention, there is provided a compound of formula I obtained by process as defined herein.

In a further aspect of the invention, there is provided a compound of formula I directly obtained by process as defined herein.

### Biological Activity

The following biological assays may be used to measure the pharmacological effects of the compounds of the present invention.

### Surface Plasmon Resonance Assay (SPR)

Biosensor experiments were conducted in filtered, degassed HEPES buffer (10 mM HEPES, 100 mM KCl, 3 mM EDTA, 0.000 05 v/v of 10% P20 BIACORE surfactant, pH 7.3) at 25° C. The 5′-biotin labeled DNA sequences (Integrated DNA Technologies) were HPLC purified and comprise the following sequences: hTel, ⁵′ biotin-d[AG₃(T₂AG₃)₃]³′; duplex, ^{5′}biotind[CGA₃T₃C₂TCTGA₃T₃CG]^{3′}. The experiments were conducted upon a BIAcore 2000 optical biosensor instrument (BIAcore Inc.). Flow cell 1 was left blank as a reference, while flow cells 2-4 were immobilized with DNA on a streptavidin-derivatized gold chip (SA chip from BIAcore) by manual injection of DNA stock solutions (flow rate of 1 μL/min) until the desired value of DNA response was obtained (350-400 RU). Typically, a series of different ligand concentrations (1 nM to 10 μM from 20 mM DMSO stock) were injected onto the chip (flow rate of 50 μL/min, 5-10 min) until a constant steady-state response was obtained followed by a dissociation period (buffer, 10 min). After every cycle, the chip surface was regenerated (20 s injection of 10 mM glycine solution, pH 2.0) followed by running buffer flow. The data were processed using BIAevaluation (BIAcore Inc.) and Kaleidagraph (Synergy Software) software for nonlinear least-squares optimization of the binding parameters.

### Growth Inhibition Assay

Growth inhibition was measured in the HT-29: human adenocarcinoma cell line using the 3-(4,5-Dimethylthiazol-2-yl)-2,5-diphenyltetrazolium bromide (MTT) assay:

Briefly, between 1500 and 2200 cells were seeded into the wells of 96-well microtiter plates and allowed to attach overnight. Test compounds were solubilised at 10 mM in dimethyl sulfoxide (DMSO), diluted in tissue culture media and added to cells at final concentrations of 30, 10, 3, 1, 0.3, 0.1, 0.03 and 0.01 μM in triplicate. Following an incubation period of 96 hr MTT was added to a final concentration of 400 μg/ml and the plates incubated for a further 4 hr. The media/MTT was then aspirated and 150 μl DMSO added. Plates were shaken briefly and the absorbance at 550 nm read using a Labtech LT-4000 plate reader. An extra plate was seeded and the MTT assay was carried out on this plate at the time of drug addition to ascertain an initial absorbance value.

Dose response curves were plotted and the GI₅₀ values (concentration required to inhibit cell growth by 50%) determined for each agent where the absorbance value for control cells minus the initial absorbance was taken as 100% growth.

Although the pharmacological properties of the compounds of the formula I vary with structural change as expected, compounds of the formula I, were found to be active in the above assays.

In general, the compounds of formula I demonstrate a quadraplex:duplex ratio in the SPR assay of greater than 2.

By way of example, the quadraplex:duplex ratio in the SPR assay for certain exemplified compounds is shown below:

| Example No. | Quadraplex:duplex ratio |
|---|---|
| 2 | 4.75 |
| 4 | 7.5 |
| 10 | 7.0 |
| 11 | 17.4 |
| 12 | >53 |
| 14 | >80 |
| 15 | 18.1 |

At the present time, the quadraplex:duplex ratio has not been assessed for all of the exemplified compounds, but in view of the structural similarity, it expected that all compounds will exhibit a quadraplex:duplex ratio of greater than 2.

In general, antiproliferative activity possessed by compounds of the formula I, may be demonstrated in the growth inhibition assay by a GI₅₀ value of less than 40 μM (preferred compounds have an GI₅₀ of less than 15 μM, more preferably less than 10 μM);

The activity for the following compounds was observed when measured in the growth inhibition assay described above:

| Example | HT 29 GI₅₀ (μM) |
|---|---|
| 1 | 10.39 |
| 2 | 0.93 |
| 3 | 3.04 |
| 4 | >30 |
| 5 | 9.88 |
| 6 | 9.39 |
| 7 | 0.95 |
| 8 | 1.04 |
| 9 | 0.95 |
| 10 | 0.27 |
| 11 | 0.76 |
| 12 | 4.49 |
| 13 | 6.17 |
| 14 | 3.67 |
| 15 | 2.99 |
| 16 | 12.75 |
| 17 | 1.88 |
| 18 | 9.66 |
| 19 | 1.03 |
| 20 | 6.46 |

### Pharmaceutical Compositions

According to a further aspect of the invention there is provided a pharmaceutical composition which comprises a compound of the invention as defined hereinbefore, or a pharmaceutically acceptable salt thereof, in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy of infection is an amount sufficient to symptomatically relieve in a warm-blooded animal, particularly a human the symptoms of infection, to slow the progression of infection, or to reduce in patients with symptoms of infection the risk of getting worse.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula I will naturally vary according to the nature and severity of the conditions, the age and sex of the animal or patient and the route of administration, according to well known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Therapeutic Uses and Applications

In one aspect, the present invention provides a compound of formula I as defined herein, or a pharmaceutically acceptable salt thereof, for use as a medicament.

The compounds of the invention are capable of inhibiting telomerase activity. Thus, in another aspect, the present invention provides a method of inhibiting telomerase activity in a cell, the method comprising administering to said cell compound of formula I as defined herein, or a pharmaceutically acceptable salt thereof.

In a further aspect, the present invention provides the use of a compound of formula I as defined herein, or a pharmaceutically acceptable salt thereof, for the inhibition of telomerase in a cell (in vivo or ex vivo).

In another aspect, the present invention provides a method of inhibiting telomerase activity in a human or animal subject in need of such inhibition, the method comprising administering to said subject an effective amount of a compound of formula I as defined herein, or a pharmaceutically acceptable salt thereof.

In another aspect, the present invention provides a compound of formula I as defined herein, or a pharmaceutically acceptable salt thereof for use in the treatment of disease or condition associated with telomerase activity.

In another aspect, the present invention provides the use of a compound of formula I as defined herein, or a pharmaceutically acceptable salt thereof, in the manufacture of a medicament for use in the treatment of disease or condition associated with telomerase activity.

In yet another aspect, the present invention provides a method of treating a proliferative disorder in a human or animal subject, the method comprising administering to said subject a therapeutically acceptable amount of a compound of formula I as defined herein, or a pharmaceutically acceptable salt thereof.

In yet another aspect, the present invention provides a compound of formula I as defined herein, or a pharmaceutically acceptable salt thereof, for use in the treatment of a proliferative disorder.

In yet another aspect, the present invention provides the use of a compound of formula I as defined herein, or a pharmaceutically acceptable salt thereof, in the manufacture of a medicament for use in the treatment of a proliferative disorder.

The term “proliferative disorder” are used interchangeably herein and pertain to an unwanted or uncontrolled cellular proliferation of excessive or abnormal cells which is undesired, such as, neoplastic or hyperplastic growth, whether in vitro or in vivo. Examples of proliferative conditions include, but are not limited to, pre-malignant and malignant cellular proliferation, including but not limited to, malignant neoplasms and tumours, cancers, leukemias, psoriasis, bone diseases, fibroproliferative disorders (e.g., of connective tissues), and atherosclerosis. Any type of cell may be treated, including but not limited to, lung, colon, breast, ovarian, prostate, liver, pancreas, brain, and skin.

The anti-proliferative effects of the compounds of the present invention have particular application in the treatment of human cancers by virtue of their telomerase inhibitory properties.

The anti-cancer effect may arise through one or more mechanisms, including but not limited to, the regulation of cell proliferation, the inhibition of angiogenesis (the formation of new blood vessels), the inhibition of metastasis (the spread of a tumour from its origin), the inhibition of invasion (the spread of tumour cells into neighbouring normal structures), or the promotion of apoptosis (programmed cell death).

The invention further provides a method of treatment of the human or animal body, the method comprising administering to a subject in need of treatment a therapeutically-effective amount of an active compound, preferably in the form of a pharmaceutical composition.

Telomerase targeting agents are also known to be useful for inhibiting cancer stem cells (see, for example, US2008/0279961).

Accordingly, the present invention also provides a method of inhibiting a cancer stem cell, the method comprising administering to said cell an effective amount of a compound of formula I as defined herein, or a pharmaceutically acceptable salt thereof.

In another aspect, the present invention provides a compound of formula I as defined herein, or a pharmaceutically acceptable salt thereof, for use in the inhibition and/or treatment of cancer stem cells.

In another aspect, the present invention provides the use of a compound of formula I as defined herein, or a pharmaceutically acceptable salt thereof, in the manufacture of a medicament for use in the inhibition and/or treatment of cancer stem cells.

### Routes of Administration

The compounds of the invention or pharmaceutical composition comprising the active compound may be administered to a subject by any convenient route of administration, whether systemically/peripherally or topically (ie., at the site of desired action).

Routes of administration include, but are not limited to, oral (e.g, by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, infraarterlal, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Combination Therapies

The antiproliferative treatment defined hereinbefore may be applied as a sole therapy or may involve, in addition to the compound of the invention, conventional surgery or radiotherapy or chemotherapy. Such chemotherapy may include one or more of the following categories of anti-tumour agents:—

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI-1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as those disclosed in International Patent Applications WO97/22596, WO 97/30035, WO 97/32856 and WO 98/13354 and compounds that work by other mechanisms (for example linomide, inhibitors of integrin αvβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) an endothelin receptor antagonist, for example zibotentan (ZD4054) or atrasentan;
(viii) antisense therapies, for example those which are directed to the targets listed above, such as ISIS 2503, an anti-ras antisense;
(ix) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(x) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a combination for use in the treatment of a cancer (for example a cancer involving a solid tumour) comprising a compound of the invention as defined hereinbefore, or a pharmaceutically acceptable salt thereof, and another anti-tumour agent.

According to this aspect of the invention there is provided a combination for use in the treatment of cancer (for example a cancer involving a solid tumour) comprising a compound of the invention as defined hereinbefore, or a pharmaceutically acceptable salt thereof, and any one of the anti-tumour agents listed under (i)-(ix) above.

In a further aspect of the invention there is provided a compound of the invention or a pharmaceutically acceptable salt thereof, for use in the treatment of cancer in combination with another anti-tumour agent, optionally selected from one listed under (i)-(ix) herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises a compound of the invention, or a pharmaceutically acceptable salt thereof, in combination with an anti-tumour agent (optionally selected from one listed under (i)-(ix) herein above), in association with a pharmaceutically acceptable diluent or carrier.

### EXAMPLES

The invention will now be illustrated in the following Examples in which, generally:

(i) Unless stated otherwise, operations were carried out at ambient temperature, i.e. in the range 17 to 25° C. and optionally under an atmosphere of an inert gas such as nitrogen or argon.
(ii) In general, the course of the reactions described herein were followed by liquid chromatography mass spectrometry (LCMS). The reaction times that are given are not necessarily the minimum attainable.
(iii) When necessary, organic solutions were dried over anhydrous magnesium sulfate, work-up procedures were carried out using traditional layer separating techniques, evaporations were carried out by rotary evaporation in vacuo.
(iv) Yields, where present, are not necessarily the maximum attainable, and when necessary, reactions were repeated if a larger amount of the reaction product was required.
(v) In general, the structures of the end-products were confirmed by nuclear magnetic resonance (NMR) and/or mass spectral techniques.
LCMS data were obtained using an Agilent 1200 series LC/MS with an Agilent 6110 quadropole MS, with Electrospray ionisation. The following two methods were used:

### (a) Polar Method:

Mobile phase A—0.1% Acetic acid in water. Mobile Phase B—0.1% in acetonitrile. Flow rate of 1.00 ml/min. Gradient from 2% B over 3 minutes to 20% B for 0.2 minutes, up to 95% B for 1.8 minutes and then back down to 2% B. Total run time of 6 minutes. Column: Phenomenex Gemini-NX 3 μm C18, 30×2.00 mm. Chromatograms based on UV detection at 254 nm. Mass Spectra were achieved using the MS in positive mode.

### (b) Alkaline Method:

Mobile phase A—0.1% ammonia solution in water. Mobile phase B—0.1% ammonia solution in acetonitrile. Flow rate of 0.500 ml/min. Gradient from 5% B over 3 minutes to 95% B, remaining at 95% B until 4.1 minutes, when it returns down to 5% B until the 7 minute run is over. Column: Waters XBridge3.5 μm C18 20×2.1 mm IS column. Chromatograms based on UV detection at 254 nm. Mass Spectra were achieved using the MS in positive mode Generally, only ions relating to the parent structure are reported.

### (c) Trial Method:

Mobile phase A—0.1% Acetic acid in water. Mobile Phase B—0.1% in acetonitrile. Flow rate of 1.00 ml/min. Gradient from 5% B rising up to 95% B over 3 minutes, remaining at 95% B for 1 minute and then back down to 5% B over 6 seconds. The total run time is 5 minutes. Column: Phenomenex Gemini-NX 3 μm C18, 30×2.00 mm. Chromatograms based on UV detection at 254 nm.
Mass Spectra were achieved using the MS in positive mode.
Proton NMR chemical shift values were measured on the delta scale at 300 or 400 MHz using a Bruker AV400. The following abbreviations have been used: s, singlet; d, doublet; t, triplet; q, quartet; m, multiplet; br, broad. Coupling constants are reported in Hz
(vi) Unless stated otherwise, compounds containing an asymmetric carbon atom were not resolved.
(vii) Intermediates were not necessarily fully purified but their structures and purity were assessed by TLC, analytical HPLC, infra-red (IR) and/or NMR analysis;
(viii) Unless otherwise stated, column chromatography (by the flash procedure) and medium pressure liquid chromatography (MPLC) were performed on Merck Kieselgel silica (Art. 9385).
(ix) The following analytical HPLC methods were used:
in general, reversed-phase silica was used with a flow rate of about 1 ml per minute and detection was by Electrospray Mass Spectrometry and by UV absorbance at a wavelength of 254 nm.
   (x) Compounds were purified by flash silica chromatography using Merck Kieselgel silica (Art. 9385).

### Example 1

### Preparation of Chromeno[4,3,2-kl]acridine

### Step 1—Preparation of 9-(2-Bromophenoxy)acridine

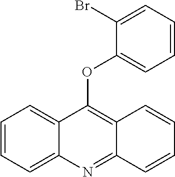 [SMILES: Brc1ccccc1Oc1c2ccccc2nc2ccccc12]

A 100 mL round bottomed flask was charged with sodium hydride (0.234 g, 4.68 mmol) under nitrogen at room temperature and diluted with anhydrous dimethylformamide (10 mL). Then a solution of 2-bromophenol (0.494 mL, 4.68 mmol) in anhydrous dimethylformamide (2 mL) was added in a slow stream over 2 minutes. After 2 hours, 9-chloroacridine (1.00 g, 4.68 mmol) was added in one portion, a condenser was fitted to the reaction and then heated to 140° C. After 3 hours, the reaction was cooled to room temperature, poured into ice water (100 mL) and then extracted with diethyl ether (4×50 mL). The combined yellow organic layers were dried over MgSO₄, filtered and concentrated in vacuo as a yellow solid. The crude material was then subjected to flash silica chromatography (5%, 10%, 15%, 20% Hexanes:Ethyl acetate), to give 1.162 g (71% yield) of the title compound as a yellow solid.

### Characterising Data:

d_{H} (DMSO-d₆): 8.25-8.27 (2H, d, J=8.8), 7.95-7.98 (2H, ddd, J=0.8, 1.3, 8.8), 7.85-7.93 (3H, m), 7.62-7.66 (2H, ddd, J=1.1, 6.9, 8.8), 7.03-7.13 (2H, m), 6.26-6.28 (1H, dd, J=1.6, 8.2).

m/z (ES⁺): 350/352 (1:1) (M+H⁺).

### Step 2—Preparation of Chromeno[4,3,2-kl]acridine

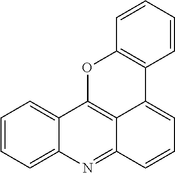 [SMILES: c1ccc2c(c1)Oc1c3ccccc3nc3cccc-2c13]

A solution of 9-(2-bromophenoxy)acridine (Step 1; 1.162 g, 3.318 mmol) in anhydrous toluene (25 mL) under nitrogen at room temperature was fitted with a condenser and heated to 130° C. Then azobisisobutyronitrile (0.054 g, 0.033 mmol) was added in one portion, followed by the addition of a solution of tributyltin hydride (1.79 mL, 6.636 mmol) in anhydrous toluene (10 mL) was added dropwise over 5 minutes. After 24, 48 and 72 hours, azobisisobutyronitrile (0.054 g, 0.033 mmol) was added in one portion, followed by the addition of a solution of tributyltin hydride (1.79 mL, 6.636 mmol) in anhydrous toluene (10 mL) was added dropwise over 5 mins. After a total of 6 days the reaction mixture was cooled to room temperature then 0° C. causing precipitation of a solid impurity. After filtering, the solvent was removed in vacuo and the crude material was subjected to flash silica chromatography (0.25%, 0.5%, 0.75%, 1%, 1.25% dichloromethane:methanol) to give 0.534 g (60% yield) of the title compound as a bright yellow solid.

### Characterising Data:

d_{H} (DMSO-d₆): 8.42-8.44 (1H, ddd, J=0.7, 1.5, 8.6), 8.29-8.31 (1H, dt, J=0.9, 8.8), 8.02-8.05 (1H, dt, J=1.0, 8.8), 7.96-7.98 (1H, dd, J=2.5, 5.5), 7.84-7.89 (3H, m), 7.55-7.63 (3H, m), 7.39-7.43 (1H, ddd, J=1.5, 7.0, 8.0).

m/z (ES⁺): 270 (M+H⁺).

### Example 2

### Preparation of 8-Methylchromeno[4,3,2-kl]acridin-8-ium chloride

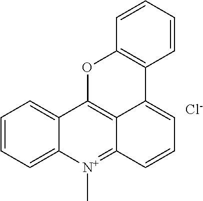 [SMILES: C[n+]1c2ccccc2c2c3c(cccc31)-c1ccccc1O2 ; [Cl-]]

A sealed tube was charged with chromeno[4,3,2-kl]acridine (prepared as described in Example 1; 0.062 g, 0.230 mmol) when iodomethane (10 mL) was added and the reaction mixture was heated to 75° C. in an oil bath behind a blast shield. After 72 hours, the reaction was allowed to cool to RT and the resulting precipitate was collected by filtration. The crude solid was then subjected to flash silica chromatography (5%, 10%, 15% dichloromethane:methanol) to yield a bright orange solid. This solid was then stirred for 16 hours with trimethylammonium chloride silica bound resin (1.16 mmol/g loading, 0.5 g), and passed through a plug of silica with 10% dichloromethane:methanol washing to give 0.044 g (60% yield) of the title compound as a bright orange solid.

### Characterising Data:

d_{H} (MeOH-d₄): 8.50-8.52 (1H, dd, J=1.6, 8.5), 8.20-8.24 (2H, t, J=8.6), 8.13-8.18 (1H, m), 8.04-8.11 (3H, m), 7.69-7.73 (1H, ddd, J=0.7, 6.7, 8.2), 7.53-7.58 (2H, m), 7.38-7.42 (1H, ddd, J=2.2, 6.0, 8.2), 4.33 (3H, s).

m/z (ES⁺): 283 (M⁺).

### Example 3

### Preparation of 8-methylthiochromeno[4,3,2-kl]acridin-8-ium chloride

### Step 1—Preparation of 9-(2-Bromophenylthio)acridine

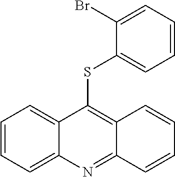 [SMILES: Brc1ccccc1Sc1c2ccccc2nc2ccccc12]

A 100 mL round bottomed flask was charged with sodium hydride (0.117 g, 2.925 mmol) under nitrogen at room temperature and diluted with anhydrous dimethylformamide (5 mL). Then a solution of 2-bromothiophenol (0.275 mL, 2.34 mmol) in anhydrous dimethylformamide (1 mL) was added in a slow stream over 1 minute. After 2 hours, 9-chloroacridine (0.5 g, 2.34 mmol) was added in one portion, a condenser was fitted to the reaction and then heated to 140° C. After 0.5 hours, the reaction was cooled to room temperature, poured into ice water (100 mL) and then extracted with diethyl ether (6×20 mL) and ethyl acetate (4×20 mL). The combined yellow organic layers were dried over MgSO₄, filtered and concentrated in vacuo as a yellow solid. The crude material was then subjected to flash silica chromatography (5%, 10% Hexanes:Ethyl acetate), to give 0.245 g (29% yield) of the title compound as a yellow solid.

### Characterising Data:

d_{H} (CHCl₃-d₁): 8.59-8.61 (2H, ddd, J=0.7, 1.2, 8.9), 8.29-8.31 (2H, d, J=8.9), 7.79-7.83 (2H, m), 7.56-7.61 (3H, m), 6.89-6.93 (1H, dt, J=1.2, 7.8), 6.78-6.82 (1H, dt, J=1.3, 7.8), 6.07-6.09 (1H, d, J=7.8).

m/z (ES⁺): 367 (M+H⁺).

### Step 2—Preparation of thiochromeno[4,3,2-kl]acridine

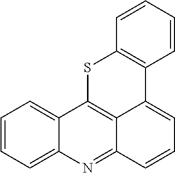 [SMILES: c1ccc2c(c1)Sc1c3ccccc3nc3cccc-2c13]

A solution of 9-(2-bromophenylthio)acridine (0.245 g, 0.669 mmol) in anhydrous toluene (20 mL) under nitrogen at room temperature was fitted with a condenser and heated to 130° C. Then azobisisobutyronitrile (0.011 g, 0.067 mmol) was added in one portion, followed by the addition of a solution of tributyltin hydride (0.361 mL, 1.338 mmol) in anhydrous toluene (6 mL) was added dropwise over 5 mins. After 24, 48 and 72 hours, azobisisobutyronitrile (0.054 g, 0.033 mmol) was added in one portion, followed by the addition of a solution of tributyltin hydride (1.79 mL, 6.636 mmol) in anhydrous toluene (10 mL) dropwise over 5 mins. After a total of 7 days the reaction mixture was cooled to room temperature then 0° C. causing precipitation of a solid impurity. After filtering, the solvent was removed in vacuo and the crude material was subjected to flash silica chromatography (5%, 10%, 20%, 50% Hexanes:Ethyl acetate) to give 0.063 g (34% yield) of the title compound as a red solid.

### Characterising Data:

d_{H} (CHCl₃-d₁): 8.04-8.09 (3H, m), 7.99-8.01 (1H, ddd, J=0.6, 1.2, 8.6), 7.90-7.92 (1H, d, J=7.3), 7.67-7.73 (2H, m), 7.40-7.44 (1H, ddd, J=1.2, 6.7, 8.6), 7.26-7.35 (3H, m).

m/z (ES⁺): 286 (M+H⁺).

### Step 3—Preparation of 8-methylthiochromeno[4,3,2-kl]acridin-8-ium chloride

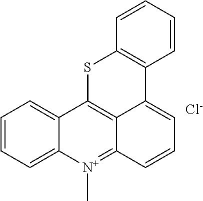 [SMILES: C[n+]1c2ccccc2c2c3c(cccc31)-c1ccccc1S2 ; [Cl-]]

A sealed tube was charged with thiochromeno[4,3,2-kl]acridine (prepared as described in Example 3; 0.013 g, 0.046 mmol) when iodomethane (9 mL) was added and the reaction mixture was heated to 75° C. in an oil bath behind a blast shield. After 5 days, the reaction was allowed to cool to RT and the resulting precipitate was collected by filtration. The crude solid was then subjected to flash silica chromatography (5%, 10%, 20% dichloromethane:methanol) to yield a red solid. This solid was then stirred for 16 hours with trimethylammonium chloride silica bound resin (1.16 mmol/g loading, 0.04 g), and passed through a plug of silica with 10% dichloromethane:methanol washing to give 0.006 g (40% yield) of the title compound as a red solid.

### Characterising Data:

d_{H} (MeOH-d₄): 8.59-8.62 (1H, d, J=7.6), 8.54-8.58 (2H, dt, J=1.2, 8.6), 8.28-8.36 (3H, m), 8.13-8.18 (1H, ddd, J=1.4, 6.8, 9.1), 7.80-7.82 (1H, dd, J=1.6, 7.6), 7.72-7.76 (1H, ddd, J=0.8, 6.9, 8.6), 7.58-7.66 (2H, m), 4.35 (3H, s).

m/z (ES⁺): 300 (M⁺).

### Example 4

### Preparation of chromeno[4,3,2-kl]acridine 8-oxide

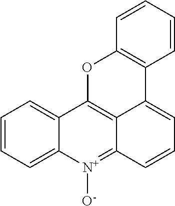 [SMILES: [O-][n+]1c2ccccc2c2c3c(cccc31)-c1ccccc1O2]

A solution of chromeno[4,3,2-kl]acridine (prepared as described in Example 1; 85 mg, 0.316 mmol) and meta-chloroperbenzoic acid (70% wt) (139 mg, 0.564 mmol, 1.78 equ.) in CHCl₃ (8.5 mL) was stirred at room temperature for three days. The reaction mixture was transferred to a separating funnel and a saturated solution of sodium bicarbonate was added. The product was extracted with dichloromethane and the combined organic extracts were washed with water, then dried over MgSO₄ and concentrated under vacuum. The product was dissolved in dichloromethane:methanol, absorbed on silica and purified by flash chromatography (gradient elution dichloromethane:methanol 0%, 2%, 4%) to give 26 mg (29% yield) of the title compound as a purple-red solid. The product was sonicated in a diethyl ether/hexane mixture, filtered, dried under suction and transferred into a vial to give 15 mg (17% yield) of the title compound.

### Characterising Data:

δ_{H} (DMSO-d₆): 8.68-8.70 (1H, d, J=9.0), 8.50-8.52 (1H, d, J=8.6), 8.39-8.41 (1H, dd, J=0.6, 9.0), 8.29-8.31 (1H, d, J=8.0), 8.07-8.08 (1H, d, J=7.5), 7.90-7.97 (2H, m), 7.72-7.76 (1H, ddd, J=1.0, 6.7, 8.6), 7.59-7.61 (2H, m), 7.39-7.43 (1H, m).

m/z (ES⁺): 286 (M⁺).

### Example 5

### Preparation of 8-(2-ethoxy-2-oxoethyl)chromeno[4,3,2-kl]acridin-8-ium iodide

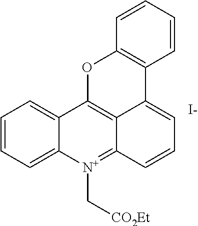 [SMILES: CCOC(=O)C[n+]1c2ccccc2c2c3c(cccc31)-c1ccccc1O2 ; [I-]]

A solution of chromeno[4,3,2-kl]acridine (prepared as described in Example 1; 30 mg, 0.111 mmol) in ethyl iodoacetate (˜500 μLE) was heated at 50° C. overnight. LCMS analysis of the crude mixture showed completion of the reaction. Dichloromethane was added to the suspension and the precipitate was filtered, washed with dichloromethane and diethyl ether to give 28 mg (52% yield) of the title compound as a bright orange solid.

### Characterising Data:

δ_{H} (DMSO-d₆): 8.95-8.97 (1H, d, J=8.4), 8.67-8.70 (1H, dd, J=1.4, 8.1), 8.62-8.63 (1H, d, J=7.5), 8.55-8.59 (1H, m), 8.35-8.36 (2H, d, J=3.1), 8.27-8.30 (1H, d, J=8.8), 8.06-8.08 (1H, dd, J=1.1, 8.4), 7.94-7.98 (1H, m), 7.86-7.90 (1H, ddd, J=1.5, 7.4, 8.6), 7.70-7.74 (1H, ddd, J=1.3, 7.3, 8.3), 6.11 (2H, s), 4.27-4.32 (2H, q, J=7.1), 1.28-1.32 (3H, t, J=7.1).

m/z (ES⁺): 356 (M⁺).

### Example 6

### Preparation of N-(chromeno[4,3,2-kl]acridin-3-yl)acetamide

### Step 1—preparation of 1-bromo-9-chloroacridine

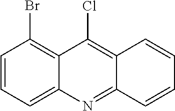 [SMILES: Clc1c2ccccc2nc2cccc(Br)c12]

2-(3-bromophenylamino)benzoic acid (6.4 g, 13.42 mmol) was refluxed in phosphorous oxychloride (35 mL) at 155° C. for 75 minutes. The solvent was then removed in vacuo and the resulting oil was diluted with dichloromethane (5 mL) and added slowly into a 1:1 mixture of dichloromethane:ammonium hydroxide, (200 mL) while stiffing at 0° C. The organic layer was removed and then aqueous layer extracted with dichloromethane (2×50 mL). The combined organic layers were dried over MgSO₄, filtered and the crude product was absorbed onto silica and subjected to flash silica chromatography. (2.5%, 5%, 10%, 12.5%, 15% Hexanes:Ethyl acetate) to yield 2 fractions, fraction 1 giving 1.2 g of the 3-bromo by-product and fraction 2 giving 1.70 g (54% yield) of the title compound.

### Characterising Data:

δ_{H} (DMSO-d₆): 8.58-8.60 (1H, d, J=8.9), 8.20-8.25 (2H, m), 8.01-8.03 (1H, dd, J=1.4, 8.3), 7.84-7.89 (1H, m), 7.66-7.74 (1H, m), 7.55-7.59 (1H, dd, J=7.6, 8.6).

m/z (ES⁺): 292 (M).

### Step 2—preparation of N-(4-(1-bromoacridin-9-yloxy)phenyl)acetamide

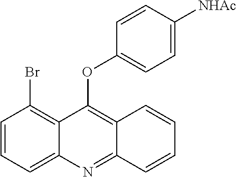 [SMILES: CC(=O)Nc1ccc(Oc2c3ccccc3nc3cccc(Br)c23)cc1]

Sodium hydride (60% in mineral oil) (15 mg, 0.384 mmol, 1.5 equ.) was added at 0° C. in one portion to a suspension of 1-bromo-9-chloroacridine (75 mg, 0.256 mmol) and acetominophene (58 mg, 0.384 mmol) in dry DMF (1 mL) and the mixture was stirred at 0° C. for 1 h. The mixture was then heated at 140° C. for 2 h. The mixture was cooled to room temperature and poured into ice. The precipitate was filtered and the solid was dried under suction overnight. LCMS analysis of the crude mixture confirmed formation of product along with ˜15% of 1-bromoacridin-9(10H)-one. The product was dissolved in dichloromethane:methanol, absorbed on silica and purified by flash chromatography (gradient elution hexanes:Ethyl acetate 50%-40%) to give 49 mg (47% yield) of the title compound as a pale yellow solid.

### Characterising Data:

δ_{H} (CHCl₃-d₁): 9.87 (1H, s), 8.26-8.28 (1H, dd, J=1.1, 8.8), 8.21-8.23 (1H, dt, J=0.9, 8.7), 7.97-7.99 (1H, m), 7.96-7.98 (1H, dd, J=1.1, 7.3), 7.88-7.92 (1H, ddd, J=1.4, 6.6, 8.7), 7.72-7.76 (1H, dd, J=7.3, 8.8), 7.58-7.62 (1H, ddd, J=1.2, 6.6, 8.8), 7.46-7.49 (2H, d, J=9.2), 6.72-6.74 (2H, d, J=9.2), 2.09 (3H, s).

### Step 3—preparation of N-(chromeno[4,3,2-kl]acridin-3-yl)acetamide

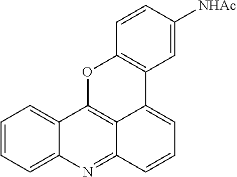 [SMILES: CC(=O)Nc1ccc2c(c1)-c1cccc3nc4ccccc4c(c13)O2]

A suspension of N-(4-(1-bromoacridin-9-yloxy)phenyl)acetamide (48 mg, 0.118 mmol) in toluene was refluxed under N₂ before the addition of azobisisobutyronitrile (10 mg, 0.061 mmol, 0.26 equ.). Tributyltin hydride (48 μL, 0.236 mmol, 2 equ.) was then added dropwise and the solution was refluxed overnight. 5-10 mg of azobisisobutyronitrile and 48 μL (0.236 mmol, 2 equ.) of tributyltin hydride were added and the mixture was refluxed for 3 h, upon which, LCMS analysis of the reaction mixture showed completion of the reaction. The mixture was concentrated under vacuum and the product was absorbed on silica. The product was purified by flash chromatography (gradient elution dichloromethane:methanol 0%, 2%, 4%, 6%) to give 19 mg (49% yield) of the title compound as a yellow/orange solid.

### Characterising Data:

δ_{H} (CHCl₃-d₁): 10.2 (1H, s), 8.47-8.48 (1H, d, J=2.4), 8.40 (1H, m), 8.02-8.05 (1H, dt, J=0.9, 8.8), 7.90-7.91 (2H, m), 7.84-7.88 (1H, m), 7.67-7.71 (2H, m), 7.87-7.62 (2H, m), 2.12 (3H, s).

m/z (ES⁺): 327 (M+H⁺).

### Example 7

### Preparation of acetamido-8-methylchromeno[4,3,2-kl]acridin-8-ium chloride

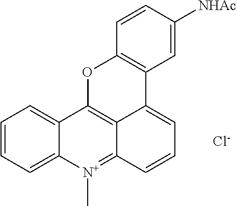 [SMILES: CC(=O)Nc1ccc2c(c1)-c1cccc3c1c(c1ccccc1[n+]3C)O2 ; [Cl-]]

A suspension of N-(chromeno[4,3,2-kl]acridin-3-yl)acetamide (18 mg, 0.0552 mmol) in methyl iodide (˜1 mL) was heated at 110° C. in a sealed tube overnight. The mixture was cooled to r.t. and diethyl ether was added. The precipitate was filtered and washed with diethyl ether to give 18 mg of an orange solid. The product was dissolved in dichloromethane:methanol and trimethylammonium chloride silica bound resin (1.16 mmol/g loading, 50 mg) was added. The mixture was stirred for ˜5 min. and then filtered over Celite®. The filtrate was concentrated under vacuum and the solid was sonicated in diethyl ether, filtered, washed with diethyl ether and air-dried to give 10 mg (50% yield) of the title compound as an orange solid.

### Characterising Data:

δₕ (CHCl₃-d₁): 10.5 (1H, s), 8.85-8.87 (1H, dd, J=1.3, 8.4)m 8.78 (1H, d, J=2.4), 8.52-8.57 (2H, m), 8.43 (1H, d, J=9.0), 8.33-8.37 (1H, ddd, J=1.5, 6.8, 9.1), 8.19-8.22 (1H, d, J=7.5), 7.95-7.98 (1H, d, J=9.0), 7.91-7.95 (1H, ddd, J=0.7, 7.1, 8.3), 7.84-7.87 (1H, dd, J=2.3, 9.0), 2.16 (3H, s).

m/z (ES⁺): 341 (M⁺).

### Example 8

### Preparation of 3-methoxy-8-methylthiochromeno[4,3,2-kl]acridin-8-ium chloride

### Step 1: Preparation of 1-bromo-9-chloro-acridine

1-bromo-9-chloro-acridine was prepared according to the procedure outlined in Example 6 (see also D. K. C. Hodgeman and R. H. Prager, Aust. J. Chem., 1972, 25, 191).

### Step 2: preparation of 1-bromo-9-(4-methoxyphenylthio)acridine

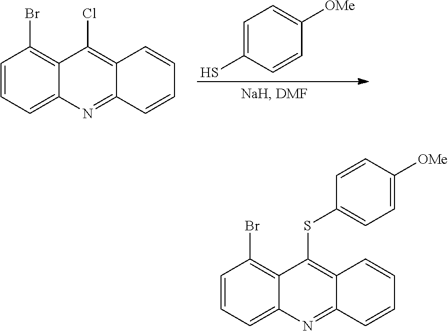

p-methoxythiophenol (462 mL, 3.76 mmol, 1.1 equ.) was added to a suspension of 1-bromo-9-chloroacridine (1 g, 3.42 mmol) in anhydrous DMF (10 mL) at 0° C. Sodium hydride (60% in mineral oil, 150 mg, 3.76 mmol, 1.1 equ.) was added in portions at 0° C. to the resulting red solution was stirred 20 min. at 0° C., then at room temperature for 1 h. The resulting suspension was filtered and the solid was washed with small amounts of DMF, then dried under suction to give 1.16 g (86%) of the title compound as an orange solid.

### Characterising Data:

δ_{H} (DMSO-d₆): 8.65-8.67 (1H, d (br), J=8.4), 8.24-8.26 (1H, dd, J=8.7, 1.2), 8.17-8.19 (1H, d(br), J=8.4), 8.09-8.11 (1H, dd, J=7.3, 1.2), 7.84-7.88 (1H, ddd, J=8.6, 6.6, 1.3), 7.70-7.74 (1H, dd, J=8.6, 7.2), 7.62-7.67 (1H, ddd, J=8.9, 6.6, 1.3), 6.89-6.91 (2H, d, J=9.0), 6.74-6.77 (2H, d, J=9.0), 3.64 (3H, s).

m/z (ES⁺): 396 (MH⁺ (⁷⁹Br)), 398 (MH⁺ (⁸¹Br)).

### Step 3: Preparation of 3-methoxy-8-methylthiochromeno[4,3,2-kl]acridin-8-ium chloride

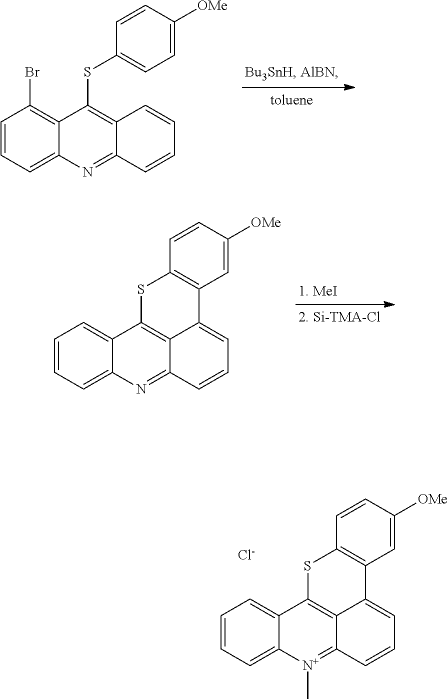 [SMILES: COc1ccc(Sc2c3ccccc3nc3cccc(Br)c23)cc1 ; COc1ccc2c(c1)-c1cccc3nc4ccccc4c(c13)S2 ; COc1ccc2c(c1)-c1cccc3c1c(c1ccccc1[n+]3C)S2 ; [Cl-]]

### 1. 3-methoxythiochromeno[4,3,2-kl]acridine

A solution of 1-bromo-9-(4-methoxyphenylthio)acridine (583 mg, 1.47 mmol) in toluene (60 mL) was refluxed under nitrogen before the addition of AIBN (24 mg, 0.147 mmol, 0.1 equ.). Tributyltin hydride (596 μL, 2.94 mmol, 2 equ.) was then added dropwise and the solution was refluxed overnight. 0.1 equ. of AIBN (24 mg) and 1 equ. of Bu₃SnH (596 μL) were added and the mixture was refluxed for a further 3 h after which, LCMS analysis of the reaction mixture showed completion of the reaction. The mixture was concentrated under vacuum and the product was dissolved in a minimal amount of DCM:MeOH 98% (the addition of a small amount of MeOH was also required). The solution was loaded on a column packed with neutralized silica gel (DCM, 1% Et₃N) and the product was purified by gradient elution (DCM:MeOH 100%-98%). Mixed fractions, which contained mainly 3-methoxythiochromeno[4,3,2-kl]acridine along with 1-bromoacridine and other uncharacterized impurities as detected by LCMS analysis, were collected and concentrated under vacuum. The resulting mixture of products was dissolved in DCM:MeOH:Et₃N and absorbed on silica. The silica absorption was loaded on a column packed with neutralized silica (Hexane:EtOAc 60%, 1% Et₃N), and the product was purified by gradient elution (Hexane:EtOAc 60%-40%). The fractions, which contained mainly 3-methoxythiochromeno[4,3,2-kl]acridine were concentrated under vacuum, then dissolved in DCM and extracted with water to remove any residual triethylammonium salts. The aqueous layer was extracted with DCM and the combined organic extracts were dried over MgSO₄ and concentrated under vacuum to give 88 mg of 3-methoxythiochromeno[4,3,2-kl]acridine as an orange/brown solid in 65% purity (as determined by LCMS analysis). The product was used without further purification in the next step.

Alternatively, 3-methoxythiochromeno[4,3,2-kl]acridine could be prepared by a palladium catalysed cyclization of 1-bromo-9-(4-methoxyphenylthio)acridine:

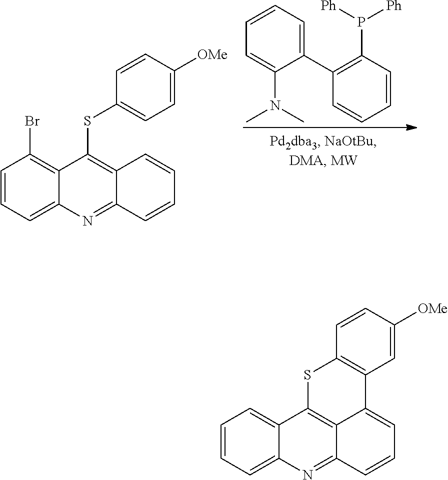

A mixture of 1-bromo-9-(4-methoxyphenylthio)acridine (300 mg, 0.757 mmol), sodium t-butoxide (87 mg, 0.908, 1.2 equ.), 2-Diphenylphosphino-2′-(N,N-dimethylamino)biphenyl (29 mg, 0.0757 mmol, 0.1 equ.) and Pd₂dba₃ (34.7 mg, 0.0378 mmol, 0.05 equ.) in DMA (3 mL) was heated at 180° C. under microwave irradiation (300 W, 300 psi) for 5 min. (hold time 5 min.). Three consecutive runs were carried out and the combined reaction mixtures were poured into ice. The precipitate was filtered and washed with water. The product was dissolved in DCM:MeOH, absorbed on silica and purified by flash chromatography (gradient elution DCM:MeOH 100%-99%-98%-97%-96%). The resulting dark brown oil/solid (293 mg) was triturated in a diethyl ether/hexane mixture and the precipitate was filtered, washed with hexane and dried under suction. The precipitate, which formed upon concentration of the filtrate, was filtered, washed with hexane to give 237 mg of 3-methoxythiochromeno[4,3,2-kl]acridine as a dark orange/brown solid (33%).

### Characterising Data:

δ_{H} (DMSO-d₆): 8.28-8.30 (1H, d, J=7.4), 8.10-8.12 (1H, ddd, J=8.6, 1.3, 0.8), 8.00-8.04 (1H, ddd, J=8.6, 1.2, 0.7), 7.98-8.00 (1H, dd, J=8.6, 0.9), 7.82-7.88 (3H, m), 7.60-7.64 (1H, ddd, J=8.6, 6.6, 1.3), 7.57-7.59 (1H, d, J=8.7), 7.12-7.15 (1H, dd, J=8.7, 2.6), 3.93 (3H, s).

### 2. 3-methoxy-8-methylthiochromeno[4,3,2-kl]acridin-8-ium chloride

A solution of 3-methoxythiochromeno[4,3,2-kl]acridine (88 mg, 0.279 mmol) in methyl iodide (4 mL) was heated in a sealed tube at 110° C. overnight, then at 120° C. for 14 h. The mixture was cooled to room temperature and concentrated under vacuum. The product was dissolved in DCM:MeOH, absorbed on silica and purified by flash chromatography (gradient elution DCM:MeOH 98%-96%-94%) to give 46 mg of a dark purple solid. The product was dissolved in DCM:MeOH and stirred with 85 mg of silica bound tetramethylammonium chloride (1.18 mmol/g, 3 equ.) for 2 h. The mixture was filtered through Celite and the filtrate was concentrated to dryness under vacuum. The product was co-evaporated with MeCN to remove any trace of MeOH, then triturated with diethyl ether, filtered and dried under suction to give 40 mg of the title compound as a dark purple solid. (7% yield from radical cyclization)

### Characterising Data:

δ_{H} (DMSO-d₆): 8.85-8.87 (1H, dd, J=4.5), 8.60-8.62 (1H, dd, J=1.1, 8.6), 8.49-8.51 (1H, d(br), J=9.0), 8.47-8.48 (2H, d(br), J=4.6), 8.25-8.30 (1H, ddd, J=1.4, 6.8, 9.1), 8.19 (1H, d, J=2.6), 8.00-8.01 (1H, d, J=8.8), 7.85-7.89 (1H, ddd, J=0.8, 6.8, 8.5), 7.41-7.44 (1H, dd, J=2.5, 8.9), 4.47 (3H, s), 4.02 (3H, s).

m/z (ES⁺): 330 (M⁺).

### Example 9

### Preparation of 4-methoxy-8-methylthiochromeno[4,3,2-kl]acridin-8-ium iodide

### Step 1: Preparation of 1-bromo-9-(3-methoxyphenylthio)acridine

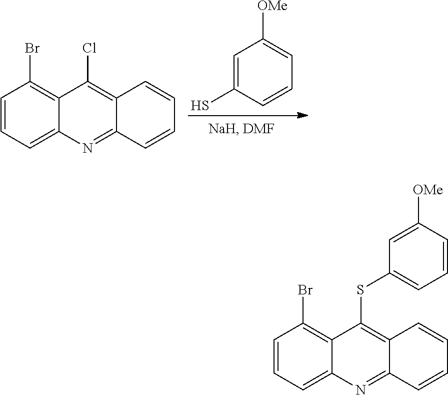

Sodium hydride (60% in mineral oil, 150 mg, 3.76 mmol, 1.1 equ.), was added in portions during a 10 minutes period to a solution of 3-methoxythiophenol (441 μL, 3.59 mmol, 1.05 equ.) in anhydrous DMF (12 mL) at 0° C. The resulting suspension was stirred at 0° C. for 10 min., then at room temperature for 1 h. 1-bromo-9-chloroacridine (1 g, 3.42 mmol; prepared as described in Examples 6) was added to the resulting solution and the mixture was heated at 140° C. for 4 h under nitrogen. The mixture was allowed to cool to room temperature and was stirred overnight. The resulting yellow suspension was filtered and the solid was triturated with hexane, filtered and dried under suction to give 850 mg of the title compound as a yellow solid. The filtrate was poured into ice and the precipitate was filtered, washed with hexane and air-dried to give 350 mg of a yellow solid. The product was dissolved in DCM, absorbed on silica and purified by flash chromatography using DCM as eluent to give 260 mg of the title compound as a yellow solid (82% combined yield).

### Characterising Data:

δᵤ (DMSO-d₆): 8.63-8.66 (1H, ddd, J=9.0, 1.3, 0.7), 8.28-8.30 (1H, dd, J=8.6, 1.2), 8.21-8.24 (1H, ddd, J=8.6, 1.2, 0.7), 8.10-8.13 (1H, dd, J=7.3, 1.2), 7.88-7.92 (1H, ddd, J=8.7, 6.6, 1.3), 7.72-7.76 (1H, dd, J=8.7, 7.2), 7.67-7.71 (1H, ddd, J=9.0, 6.6, 1.3), 7.05-7.09 (1H, dd, J1=J2=8.0), 6.67-6.70 (1H, ddd, J=8.3, 2.5, 0.8), 6.49-6.50 (1H, dd, J1=J2=2.1), 6.30-6.33 (1H, ddd, J=7.8, 1.8, 0.9), 3.60 (3H, s).

m/z (ES⁺): 398 (MH⁺, ⁸¹Br), 396 (MH⁺, ⁷⁹Br).

### Step 2: Preparation of 4-methoxythiochromeno[4,3,2-kl]acridine

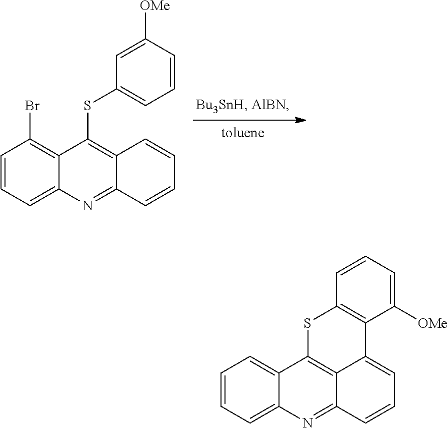 [SMILES: COc1cccc(Sc2c3ccccc3nc3cccc(Br)c23)c1 ; COc1cccc2c1-c1cccc3nc4ccccc4c(c13)S2]

A solution of 1-bromo-9-(3-methoxyphenylthio)acridine (850 mg, 2.15 mmol) and tributyltin hydride (1.15 mL, 4.29 mmol, 2 equ.) in anhydrous toluene (80 mL) was stirred at 140° C. under nitrogen before the addition of AIBN (35 mg, 0.215 mmol, 0.1 equ.). The resulting hazy solution was stirred at 140° C. for 40 min., and was then allowed to cool to room temperature. The mixture was concentrated under vacuum and the product was absorbed on silica and purified by flash chromatography (gradient elution DCM:MeOH 100:1-50:1-100% MeOH) to give 13 mg (2%) of the title compound as an orange solid.

### Characterising Data:

δ_{H} (DMSO-d₆): 8.82-8.84 (1H, d, J=7.4), 8.09-8.11 (1H, d, J=8.6), 7.99-8.02 (1H, d, J=8.6), 7.92-7.94 (1H, d, J=8.5), 7.81-7.85 (2H, dd, J=7.9, 7.8), 7.59-7.62 (1H, dd, J1=J2=7.7), 7.39-7.43 (1H, dd, J1=J2=8.0), 7.20-7.22 (1H, d, J=7.6), 7.15-7.17 (1H, d, J=8.2), 3.98 (3H, s).

m/z (ES⁺): 316 (MH⁺).

### Step 3: Preparation of 4-methoxy-8-methylthiochromeno[4,3,2-kl]acridin-8-ium iodide

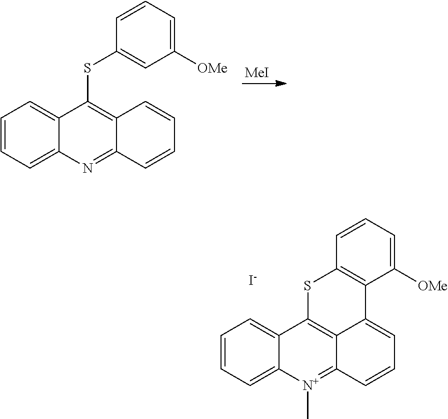 [SMILES: "MeI" ; COc1cccc(Sc2c3ccccc3nc3ccccc23)c1 ; COc1cccc2c1-c1cccc3c1c(c1ccccc1[n+]3C)S2 ; [I-]]

A solution of 4-methoxythiochromeno[4,3,2-kl]acridine (10 mg, 0.0317 mmol) in methyl iodide (3 mL) was heated in a sealed tube at 90° C. for 1 hour, then at 50° C. for 7 days. The reaction mixture was allowed to cool to room temperature and was concentrated under vacuum. The product was dissolved in DCM:MeOH, absorbed on silica and purified by flash chromatography (gradient elution DCM:MeOH 100:1-50:1-20:1-10:1) to give 4.2 mg (40%) of the title compound as a red solid.

### Characterising Data:

δ_{H} (MeOD): 9.39-9.41 (1H, dd, J=7.7, 1.2), 8.64-8.67 (1H, dd, J=8.6, 1.2), 8.38-8.42 (2H, m), 8.34 (1H, dd, J=9.0, 1.2), 8.23-8.27 (1H, ddd, J=9.0, 6.8, 1.4), 7.81-7.85 (1H, ddd, J=8.5, 6.9, 0.9), 7.63-7.68 (1H, dd, J=8.1), 7.49-7.51 (1H, dd, J=7.9, 1.1), 7.39-7.41 (1H, dd, J=8.3, 0.8), 4.51 (3H, s), 4.11 (3H, s).

### Example 10

### Preparation of 3,10-dimethoxy-8-methylthiochromeno[4,3,2-kl]acridin-8-ium chloride

### Step 1: Preparation of methyl 2-(3-bromophenylamino)-4-methoxybenzoate

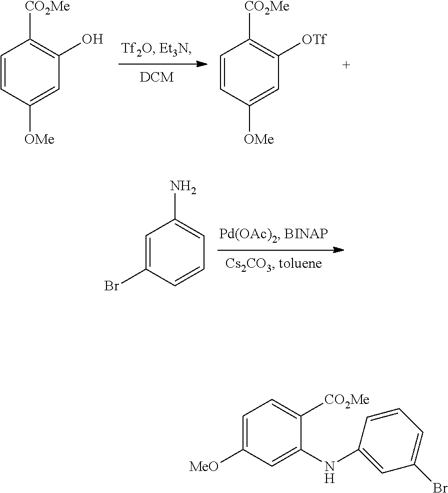 [SMILES: COC(=O)c1ccc(OC)cc1O ; COC(=O)c1ccc(OC)cc1OS(=O)(=O)C(F)(F)F ; Nc1cccc(Br)c1 ; COC(=O)c1ccc(OC)cc1Nc1cccc(Br)c1]

Triethylamine (7.64 mL, 54.8 mmol, 2 equ.) followed by triflic anhydride (5.54 mL, 32.9 mmol, 1.2 equ.) were added to a solution of methyl 4-methoxy,2-hydroxybenzoate (5 g, 27.4 mmol) in DCM (100 mL) at −78° C. and the mixture was stirred at −78° C. for 30 min., then at room temperature for 1 h after which, TLC and LCMS analyses showed completion of the reaction. The reaction was quenched with water and the product was extracted with DCM. The combined organic extracts were washed successively with 1N HCl, sat.NaHCO₃ and brine. The extracts were dried over MgSO₄ and concentrated under vacuum to give 8.81 g of methyl 4-methoxy-2-(trifluoromethylsulfonyloxy)benzoate as a dark brown oil. The product was used without further purification for the next step.

Nitrogen was bubbled through a solution of crude methyl 4-methoxy-2-(trifluoromethylsulfonyloxy)benzoate (8.81 g, 27.4 mmol), 3-bromoaniline (3.58 mL, 32.9 mmol, 1.2 equ.) and cesium carbonate (11.6 g, 35.6 mmol, 1.3 equ.) before the addition of palladium acetate (308 mg, 1.37 mmol, 0.05 equ.) and BINAP (1.7 g, 2.74 mmol, 0.10 equ.). The mixture was heated at 120° C. under nitrogen overnight and was allowed to cool to room temperature. The reaction mixture was filtered and the filtrate was concentrated under vacuum. The residue was dissolved in DCM, absorbed on silica and the product was purified by flash chromatography (gradient elution Hexane:EtOAc 97.5%-95%-92.5%) to give 3.8 g of the title compound as a pale yellow oil (41% over two steps).

### Characterising Data:

δ_{H} (CDCl₃): 9.66 (1H, s (br)), 7.93-7.95 (1H, d, J=8.9), 7.45-7.46 (1H, m), 7.19-7.23 (3H, m), 6.74-6.75 (1H, d, J=2.5), 6.36-6.39 (1H, dd, J=8.9, 2.5), 3.89 (3H, s), 3.79 (3H, s).

### Step 2: Preparation of 2-(3-bromophenylamino)-4-methoxybenzoic acid

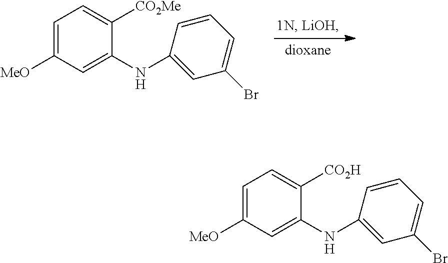 [SMILES: COC(=O)c1ccc(OC)cc1Nc1cccc(Br)c1 ; COc1ccc(C(=O)O)c(Nc2cccc(Br)c2)c1]

A solution of methyl 2-(3-bromophenylamino)-4-methoxybenzoate (4 g, 11.9 mmol) in dioxane:1N LiOH (4:1, 125 mL) was refluxed overnight. The reaction mixture was cooled to room temperature, and concentrated under vacuum. 3N HCl was added and the precipitate was filtered, then washed with water. The solid was dried in a vacuum-oven overnight to give 3.62 g (95%) of the title compound as a white solid.

### Characterising Data:

δ_{H} (DMSO-d₆): 12.9 (1H, s (br)), 9.83 (1H, s (br)), 7.87-7.89 (1H, d, J=8.9), 7.45-747 (1H, m), 7.30-7.33 (2H, m), 7.22-7.25 (1H, m), 6.70-6.71 (1H, d, J=2.4), 6.45-6.48 (1H, dd, J=8.9, 2.4), 3.75 (3H, s).

m/z (ES⁺): 324 (MH⁺ (⁸¹Br)), 322 (MH⁺ (⁷⁹Br)).

Note: The product, which was 90 to 95% pure as estimated by NMR analysis, was used without further purification in the next step.

### Step 3: Preparation of 1-bromo-9-chloro-6-methoxyacridine

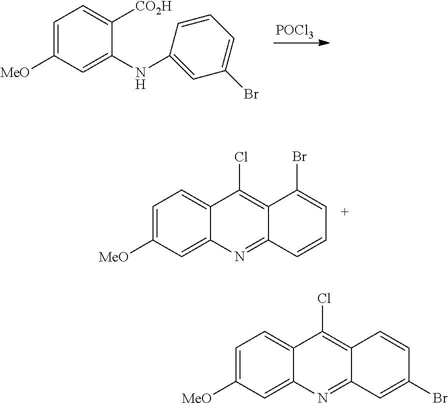

A solution of 2-(3-bromophenylamino)-4-methoxybenzoic acid (3.6 g, 11.2 mmol) in phosphorus oxychloride (15 mL) was heated at 120° C. for 2 h, then concentrated to dryness by distillation. The resulting black residue was dissolved in chloroform and poured into an ice/cc. aq.NH₃ mixture. The product was extracted with chloroform and the combined organic extracts were dried over MgSO₄. The resulting solid was dissolved in DCM and absorbed on silica. 1.05 g of the title compound was separated from 3-bromo-9-chloro-6-methoxyacridine by flash chromatography (gradient elution Hexane:EtOAc 90%-80%-70%-60%-50%).

### Characterising Data:

δ_{H} (DMSO-d₆): 8.41-8.44 (1H, d, J=7.4), 8.14-8.16 (1H, dd, J=8.6, 1.2), 8.05-8.07 (1H, dd, J=7.4, 1.2), 7.68-7.71 (1H, dd, J=8.6, 7.4), 7.49-7.51 (1H, dd, J=7.8, 2.6), 7.49 (1H, s), 4.02 (3H, s).

### Step 4: Preparation of 1-bromo-6-methoxy-9-(4-methoxyphenylthio)acridine

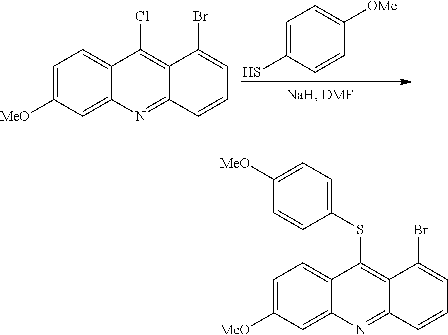

4-methoxythiophenol (74 μL, 0.597 mmol, 1.1 equ.) was added to a suspension of 1-bromo-9-chloro-6-methoxyacridine (175 mg, 0.542 mmol) in anhydrous DMF (2 mL) at 0° C. Sodium hydride (60% in mineral oil, 24 mg, 0.597 mmol, 1.1 equ.) was added in portions to the solution and the mixture was stirred at 0° C. for 30 min., then at room temperature for 1 h. The resulting yellow suspension was poured into ice and the precipitate was filtered, washed with water, dried under suction then in a vacuum-oven overnight to give 229 mg of the title compounds as a yellow solid (99%).

### Characterising Data:

δ_{H} (DMSO-d₆): 8.55-8.58 (1H, d, J=9.6), 8.14-8.17 (1H, dd, J=8.6, 1.2), 8.00-8.02 (1H, dd, J=7.3, 1.2), 7.66-7.70 (1H, dd, J=8.6, 7.3), 7.45-7.46 (1H, d, J=2.5), 7.30-7.33 (1H, dd, J=9.6, 2.5), 6.87-6.89 (2H, d, J=9.1), 6.75-6.77 (2H, d, J=9.1), 3.98 (3H, s), 3.65 (3H, s).

m/z (ES⁺): 428 (MH⁺ (⁸¹Br)), 426 (MH⁺ (⁷⁹Br)).

### Step 5: Preparation of 3,10-dimethoxythiochromeno[4,3,2-kl]acridine

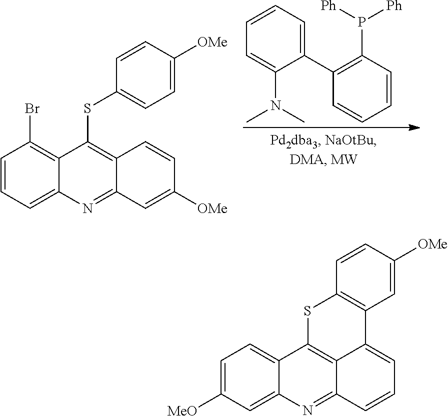

A mixture of 1-bromo-6-methoxy-9-(4-methoxyphenylthio)acridine (100 mg, 0.234 mmol), sodium t-butoxide (26 mg, 028 mmol, 1.2 equ.), 2-Diphenylphosphino-2′-(N,N-dimethylamino)biphenyl (9 mg, 0.023 mmol, 0.1 equ.) and Pd₂dba₃ (11 mg, 0.0012 mmol, 0.05 equ.) in anhydrous DMA (1 mL) was heated at 180° C. under microwave irradiation (300 W, 300 psi) for 5 min. (hold time 5 min.). The reaction mixtures from two separate runs carried out on 50 mg and 100 mg scale were combined and the mixture was concentrated under vacuum. The residue was dissolved in DCM:MeOH and the product was absorbed on silica, then purified by flash chromatography (gradient elution DCM:MeOH 100%-98%-97%-96%) to give 68 mg of the title compound as an orange solid (34%).

### Characterising Data:

δ_{H} (DMSO-d₆): 8.22-8.24 (1H, d, J=7.4), 7.99-8.01 (1H, d, J=9.3), 7.89-7.91 (1H, dd, J=8.6, 1.0), 7.85-7.91 (1H, d, J=2.6), 7.80-7.84 (1H, dd, J=8.6, 7.4), 7.53-7.56 (1H, d, J=8.6), 7.31-7.32 (1H, d, J=2.6), 7.25-7.28 (1H, dd, J=9.3, 2.6), 7.10-7.13 (1H, dd, J=8.7, 2.6), 3.97 (3H, s), 3.92 (3H, s).

m/z (ES⁺): 346 (MH⁺)

### Step 6: Preparation of 3,10-dimethoxy-8-methylthiochromeno[4,3,2-kl]acridin-8-ium chloride

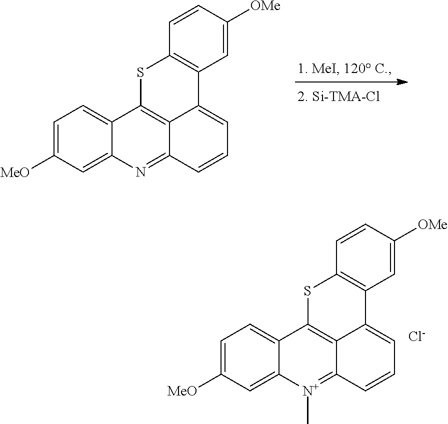 [SMILES: COc1ccc2c(c1)-c1cccc3nc4cc(OC)ccc4c(c13)S2 ; COc1ccc2c(c1)-c1cccc3c1c(c1ccc(OC)cc1[n+]3C)S2 ; [Cl-]]

A mixture of 3,10-dimethoxythiochromeno[4,3,2-kl]acridine (15 mg, 0.046 mmol) in methyl iodide (2 mL) was heated at 120° C. in a sealed tube for 2 days after which, and was then allowed to cool to room temperature. The mixture was concentrated to dryness under vacuum, and the product was dissolved in DCM:MeOH. Silica bound tetramethylammonium chloride (118 mg) (1.18 mmol/g, 3 equ.) was added and the mixture was stirred for 3 h. The mixture was filtered over Celite and the filtrate was concentrated under vacuum. The resulting solid was triturated with diethyl ether, filtered and dried under suction to give 14 mg of the title compound as a dark purple solid (78%).

### Characterising Data:

δ_{H} (DMSO-d₆): 8.75-8.77 (1H, dd, J=6.4, 2.2), 8.43-8.45 (1H, d, J=9.4), 8.47-8.39 (2H, m), 8.10-8.11 (1H, d, J=2.3), 7.88-7.90 (1H, d, J=8.8), 7.56-7.57 (1H, d, J=2.3), 7.46-7.49 (1H, dd, J=9.4, 2.3), 7.35-7.38 (1H, dd, J=8.8, 2.4), 4.38 (3H, s), 4.16 (3H, s), 3.99 (3H, s).

### Example 11

### Preparation of 10-methoxy-2-(methoxycarbonyl)-8-methylthiochromeno[4,3,2-kl]acridin-8-ium chloride

### Step 1: Preparation of methyl 3-mercaptobenzoate

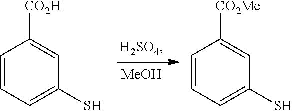 [SMILES: "MeOH" ; O=C(O)c1cccc(S)c1 ; COC(=O)c1cccc(S)c1]

A mixture of 3-mercaptobenzoic acid (1.52 g, 9.86 mmol) and sulfuric acid (3 mL) was refluxed in methanol (30 mL) for 4 h. The mixture was concentrated to a small volume under vacuum and diethyl ether, followed by water were added. The product was extracted with diethyl ether and the combined organic extracts were washed successively with sat. NaHCO₃ and brine, then dried over MgSO₄ and concentrated under vacuum to give 1.28 g of the title compound as a pale yellow oil (77%).

### Step 2: Preparation of methyl 10-methoxythiochromeno[4,3,2-kl]acridine-2-carboxylate and methyl 10-methoxythiochromeno[4,3,2-kl]acridine-4-carboxylate

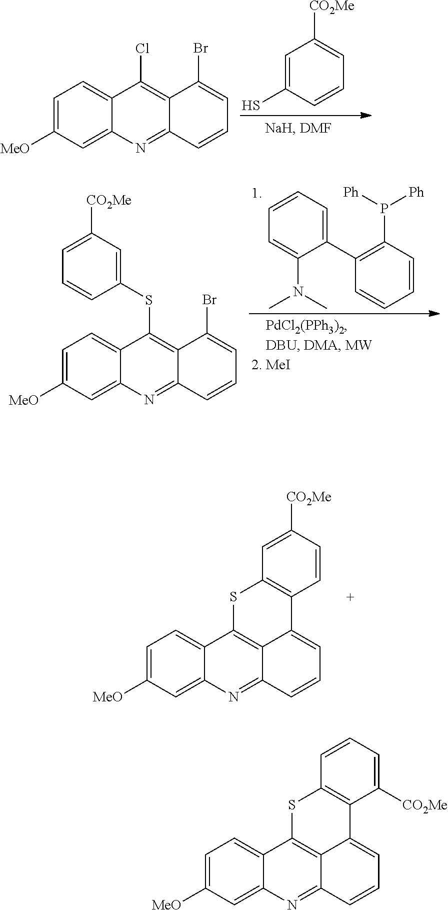

A solution of methyl 3-mercaptobenzoate (110 mg, 0.651 mmol, 1.05 equ.) in anhydrous DMF (2 mL) was added dropwise at 0° C. to a suspension of 1-bromo-9-chloro-6-methoxyacridine (200 mg, 0.62 mmol) in anhydrous DMF. The resulting red solution was stirred for 5 min. before the addition of sodium hydride (60% in mineral oil, 26 mg, 0.651 mmol, 1.05 equ.). The mixture was stirred at 0° C. for 30 min., then at room temperature for 1 h. The resulting yellow suspension was poured into ice and the precipitate was filtered and dried under suction to give 245 mg of methyl 3-(1-bromo-6-methoxyacridin-9-ylthio)benzoate as a yellow solid (85%). The product, which was 80 to 85% pure as estimated by NMR and LCMS analyses, was used without further purification in the next step. (m/z (ES⁺): 456 (MH⁺ (⁸¹Br)), 454 (MH⁺ (⁷⁹Br))).

A mixture of methyl 3-(1-bromo-6-methoxyacridin-9-ylthio)benzoate (195 mg, 0.429 mmol), DBU (320 μL, 0.21 mmol, 5 equ.), 2-Diphenylphosphino-2′-(N,N-dimethylamino)biphenyl (17 mg, 0.043 mmol, 0.1 equ.), and PdCl₂(PPh₃)₂ (16 mg, 0.021 mmol, 0.05 equ.) in DMA (2 mL) was heated at 180° C. under microwave irradiation (300 W, 300 psi) for 5 min. (hold time 5 min.). Methyl iodide (500 μL, 8.03 mmol, 18 equ.) was added and the reaction mixture was stirred overnight. 1N HCl (1.7 mL, 4 equ.) was added dropwise and the reaction mixture from a separate experiment carried out on 50 mg scale was combined. The resulting mixture was poured into ice and the precipitate was filtered then dried under suction overnight. The resulting solid was dissolved in DCM:MeOH, absorbed on silica and the products were purified by flash chromatography (gradient elution DCM:MeOH 100%-98%-97%-96%).

The first isolated yellow/orange solid (55 mg) corresponded to the C4 regioisomer and was 80% pure as estimated by LCMS and NMR analyses. The product was used without further purification in the next step.

### Characterising Data:

δ_{H} (DMSO-d₆): 8.03-8.06 (1H, d, J=9.3), 7.95-7.97 (1H, dd, J=8.6, 0.96), 7.82-7.87 (1H, dd, J=8.7, 7.4), 7.81-7.84 (1H, dd, J=7.6, 1.8), 7.58 (1H, d, J=1.8), 7.55-7.57 (1H, d, J=7.5), 7.36-7.38 (2H, m), 7.30-7.33 (1H, dd, J=9.3, 2.5), 3.99 (3H, s), 3.81 (3H, s).

m/z (ES⁺): 374 (MH⁺)

The second isolated orange solid (36 mg) corresponded to the C2 regioisomer. (45% combined yield)

### Characterising Data:

δ_{H} (DMSO-d₆): 8.46-8.48 (1H, d, J=8.7), 8.25 (1H, m), 8.06 (1H, s (br)), 7.85-7.99 (4H, m), 7.29-7.31 (2H, m), 3.98 (3H, s), 3.92 (3H, s).

m/z (ES⁺): 374 (MH⁺)

### Step 3: Preparation of 10-methoxy-2-(methoxycarbonyl)-8-methylthiochromeno[4,3,2-kl]acridin-8-ium chloride

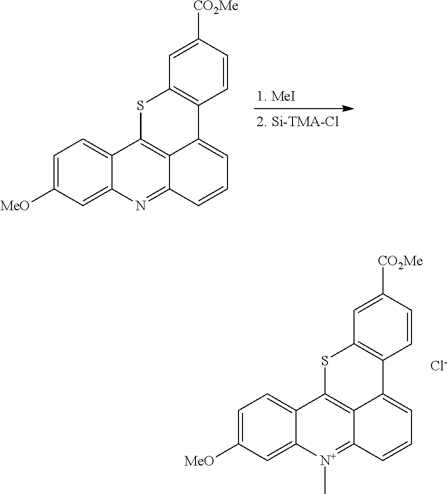 [SMILES: COC(=O)c1ccc2c(c1)Sc1c3ccc(OC)cc3nc3cccc-2c13 ; COC(=O)c1ccc2c(c1)Sc1c3ccc(OC)cc3[n+](C)c3cccc-2c13 ; [Cl-]]

A suspension of methyl 10-methoxythiochromeno[4,3,2-kl]acridine-2-carboxylate (36 mg, 0.0964 mmol) in methyl iodide (5 mL) was heated at 120° C. in a sealed tube overnight and was allowed to cool to room temperature. The mixture was concentrated to dryness under vacuum and the resulting solid was dissolved in 90% DCM:MeOH. 220 mg of silica bound tetramethylammonium chloride (1.18 mmol/g, 2.7 equ.) was added and the mixture was stirred overnight, then filtered over Celite. The filtrate was concentrated to dryness under vacuum and the resulting solid was triturated with diethyl ether, filtered and dried under suction to give 45 mg of the title compound as a red/orange solid (109%).

### Characterising Data:

δ_{H} (DMSO-d₆): 8.71-8.72 (1H, d, J=8.8), 8.67-8.69 (1H, d, J=7.6), 8.45-8.47 (1H, d, J=8.6), 8.37-8.39 (1H, d, J=9.4), 8.37-8.38 (1H, d, J=7.7), 8.34 (1H, d, J=1.7), 8.04-8.07 (1H, dd, J=8.6, 1.8), 7.56 (1H, d, J=2.3), 7.50-7.53 (1H, dd, J=9.4, 2.3), 4.41 (3H, s), 4.17 (3H, s), 3.96 (3H, s).

m/z (ES⁺): 388 (M+)

Note: The structure was confirmed by COSY and NOESY analyses.

### Example 12

### Preparation of 10-methoxy-4-(methoxycarbonyl)-8-methylthiochromeno[4,3,2-kl]acridin-8-ium chloride

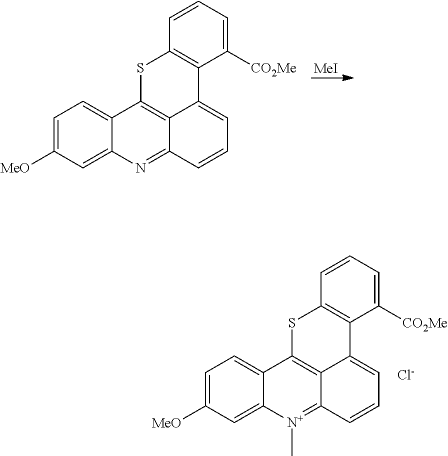 [SMILES: "MeI" ; COC(=O)c1cccc2c1-c1cccc3nc4cc(OC)ccc4c(c13)S2 ; COC(=O)c1cccc2c1-c1cccc3c1c(c1ccc(OC)cc1[n+]3C)S2 ; [Cl-]]

A suspension of methyl 10-methoxythiochromeno[4,3,2-kl]acridine-2-carboxylate (55 mg, 0.147 mmol; Example 11 above) in methyl iodide (5 mL) was heated at 120° C. in a sealed tube overnight. The mixture was concentrated to dryness under vacuum and the resulting solid was dissolved in DCM:MeOH, absorbed on silica and purified by flash chromatography (gradient elution DCM:MeOH 98%-95%). The product was dissolved in DCM:MeOH and 220 mg of silica bound tetramethylammonium chloride (1.18 mmol) were added. The mixture was stirred overnight and then filtered over Celite. The filtrate was concentrated under vacuum and the product was triturated with diethyl ether, filtered and dried under suction to give 47 mg of the title compound as a red/orange solid (76%).

### Characterising Data:

δ_{H} (DMSO-d₆): 8.55-8.57 (1H, d, J=8.7), 8.54-8.57 (1H, d, J=9.4), 8.40-8.44 (1H, dd, J=9.0, 7.7), 8.17-8.19 (1H, dd, J=7.9, 1.4), 7.88-7.90 (1H, dd, J=7.5, 1.4), 7.83-7.84 (1H, d, J=7.8), 7.80-7.84 (1H, dd, J=7.7), 7.67-7.68 (1H, d, J=2.3), 7.57-7.60 (1H, dd, J=9.4, 2.2), 4.49 (3H, s), 4.20 (3H, s), 3.85 (3H, s).

m/z (ES⁺): 388 (M+)

Note: The structure was confirmed by COSY and NOESY analyses.

### Example 13

### Preparation of 3-acetoxy-8-methylthiochromeno[4,3,2-kl]acridin-8-ium chloride

### Step 1: Preparation of 4-(1-bromoacridin-9-ylthio)phenol

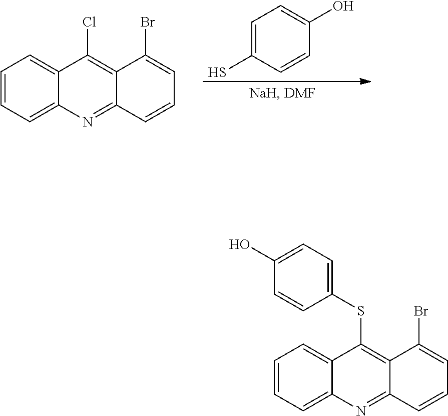 [SMILES: Clc1c2ccccc2nc2cccc(Br)c12 ; Oc1ccc(S)cc1 ; Oc1ccc(Sc2c3ccccc3nc3cccc(Br)c23)cc1]

4-mercaptophenol (45 mg, 0.359 mmol, 1.05 equ.) was added in one portion to a suspension of 1-bromo-9-chloroacridine (100 mg, 0.342 mmol; see Example 6) in anhydrous DMF (1 mL) at 0° C. and the resulting red mixture was stirred for 5-10 min. at 0° C. before the addition of sodium hydride (60% in mineral oil, 15 mg, 0.359 mmol, 1.05 equ.). The mixture was stirred at 0° C. for 30 min., then at room temperature for 1 h. The resulting bright orange solution was poured into ice and the precipitate was filtered, washed with water and dried in a vacuum-oven overnight to give 101 mg of the title compound as an orange solid. (77%)

### Characterising Data:

δ_{H} (DMSO-d₆): 9.53 (1H, s), 8.64-8.67 (1H, ddd, J=8.9, 1.2, 0.6), 8.22-8.25 (1H, dd, J=8.7, 1.2), 8.15-8.17 (1H, ddd, J=8.7, 1.2, 0.6), 8.08-8.10 (1H, dd, J=7.2, 1.2), 7.82-7.87 (1H, ddd, J=8.6, 6.6, 1.2), 7.69-7.73 (1H, dd, J=8.6, 7.3), 7.61-7.65 (1H, ddd, J=8.9, 6.6, 1.2), 6.80-6.82 (2H, d, J=8.6), 6.55-6.58 (2H, d, J=8.6).

### Step 2: Preparation of Thiochromeno[4,3,2-kl]acridin-3-ol

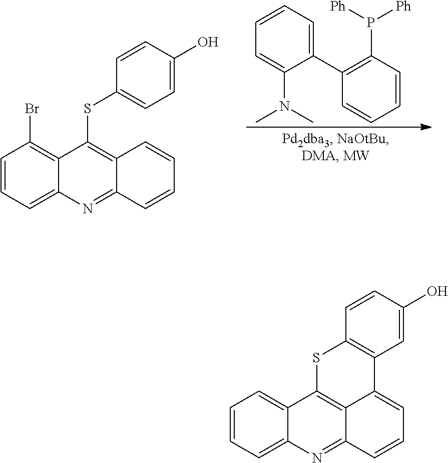 [SMILES: Oc1ccc(Sc2c3ccccc3nc3cccc(Br)c23)cc1 ; CN(C)c1ccccc1-c1ccccc1P(c1ccccc1)c1ccccc1 ; Oc1ccc2c(c1)-c1cccc3nc4ccccc4c(c13)S2]

A mixture of 4-(1-bromoacridin-9-ylthio)phenol (380 mg, 0.998 mmol), sodium t-butoxide (107 mg, 1.097 mmol, 1.1 equ.), 2-Diphenylphosphino-2′-(N,N-dimethylamino)biphenyl (38 mg, 0.0998 mmol, 0.1 equ.) and Pd₂dba₃ (46 mg, 0.0495 mmol, 0.05 equ.) in DMA (4 mL) was heated at 180° C. under microwave irradiation (300 W, 300 psi) for 5 min. (hold time 5 min.). The reaction mixtures from three separate experiments carried out on 300 mg scale were combined and the mixture was poured into ice. The resulting precipitate was filtered, washed with water and dried under suction. The product was dissolved in DCM:MeOH, absorbed on silica and purified by flash chromatography (gradient elution DCM:MeOH 98%-97%-96%-94%-92%). The resulting light brown solid was triturated with diethyl ether, filtered, washed with diethyl ether and dried under suction to give 184 mg of the title compound as a light brown solid (18%).

Alternatively, the title compound could be prepared by demethylation of 3-methoxythiochromeno[4,3,2-kl]acridine:

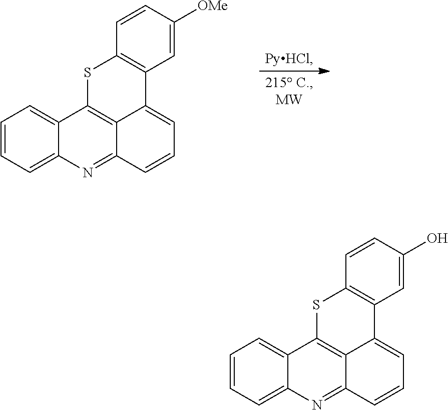 [SMILES: COc1ccc2c(c1)-c1cccc3nc4ccccc4c(c13)S2 ; Oc1ccc2c(c1)-c1cccc3nc4ccccc4c(c13)S2]

A mixture of 3-methoxythiochromeno[4,3,2-kl]acridine (101 mg, 0.320 mmol) and pyridine hydrochloride (200 mg) was heated at 215° C. under microwave irradiation (300 W, 100 psi, 5 min. hold time) for 5 min. Ice/water was added to the reaction mixture and the precipitate was filtered, washed successively with water and sat. NaHCO₃ and dried under suction. The resulting solid was dissolved in DCM:MeOH, absorbed on silica and purified by flash chromatography (gradient elution DCM:MeOH 98%-96%-94%-92%) to give 35 mg of the title compound as an orange solid (36%).

### Characterising Data:

δ_{H} (DMSO-d₆): 10.0 (1H, s), 8.09-8.12 (1H, ddd, J=8.6, 1.2, 0.6), 8.05-8.07 (1H, dd, J=6.8, 0.8), 8.00-8.03 (1H, ddd, J=8.6, 1.1, 0.7), 7.95-7.98 (1H, dd, J=8.6, 0.9), 7.84-7.87 (1H, dd, J=7.8, 6.8), 7.81-7.84 (1H, dd, J=8.7, 1.4), 7.73-7.74 (1H, d, J=2.3), 7.59-7.63 (1H, ddd, J=8.6, 6.6, 1.2), 7.47-7.49 (1H, d, J=8.6), 6.96-6.99 (1H, dd, J=8.6, 2.4),

m/z (ES⁺): 302 (MH⁺)

### Step 3: Preparation of 3-acetoxy-8-methylthiochromeno[4,3,2-kl]acridin-8-ium chloride

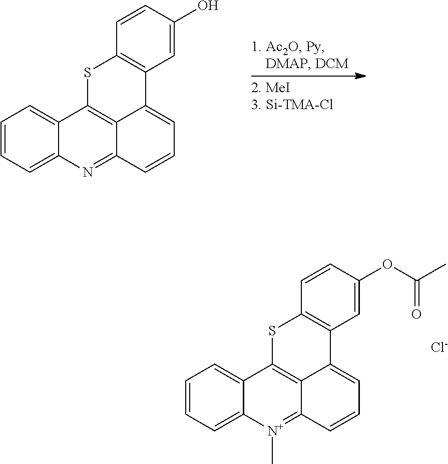 [SMILES: Oc1ccc2c(c1)-c1cccc3nc4ccccc4c(c13)S2 ; CC(=O)Oc1ccc2c(c1)-c1cccc3c1c(c1ccccc1[n+]3C)S2 ; [Cl-]]

Acetic anhydride (20 μL, 0.214 mmol, 1.5 equ.) was added to a suspension of thiochromeno[4,3,2-kl]acridin-3-ol (43 mg, 0.143 mmol), pyridine (15 μL, 0.186 mmol, 1.3 equ.) and DMAP (catalytic amount) in DCM (4 mL) and the mixture was stirred overnight. Water was added and the layers were separated. The aqueous layer was extracted with DCM and the combined organic extracts were washed with sat. NaHCO₃, dried over MgSO₄ and concentrated under vacuum to give 50 mg of thiochromeno[4,3,2-kl]acridin-3-yl acetate. The product was used without further purification in the next step.

A suspension of thiochromeno[4,3,2-kl]acridin-3-yl acetate (50 mg, 0.146 mmol) in methyl iodide (4 mL) was heated at 100° C. in a sealed tube overnight. The reaction mixture was cooled to room temperature, then concentrated under vacuum. The resulting dark purple solid was triturated with diethyl ether, filtered, washed with diethyl ether and dried under suction. The resulting dark purple solid (53 mg) was dissolved in DCM:MeOH and 90 mg of silica bound tetramethylammonium chloride (1.18 mmol/g) were added. The mixture was stirred overnight, then filtered through Celite. The filtrate was concentrated under vacuum and the resulting solid was dissolved in DCM:MeOH, absorbed on silica and purified by flash chromatography (gradient elution DCM:MeOH 98%-96%-94%-92%-90%). The product was then triturated with diethyl ether, filtered, washed with diethyl ether and dried under suction to give 16 mg of the title compound as a dark purple solid (29% over two steps).

### Characterising Data:

δ_{H} (DMSO-d₆): 8.77-8.79 (1H, d, J=7.4), 8.64-8.66 (1H, dd, J=8.4, 1.2), 8.62 (1H, d, J=2.1), 8.47-8.56 (3H, m), 8.29-8.33 (1H, ddd, J=8.6, 6.9, 1.4), 8.11-8.13 (1H, d, J=8.6), 7.89-7.93 (1H, dd, J=8.6, 7.1), 7.59-7.62 (1H, dd, J=8.6, 2.2), 4.51 (3H, s), 2.39 (3H, s).

m/z (ES⁺): 358 (M⁺)

### Example 14

### Preparation of 3-hydroxy-8-methylthiochromeno[4,3,2-kl]acridin-8-ium chloride

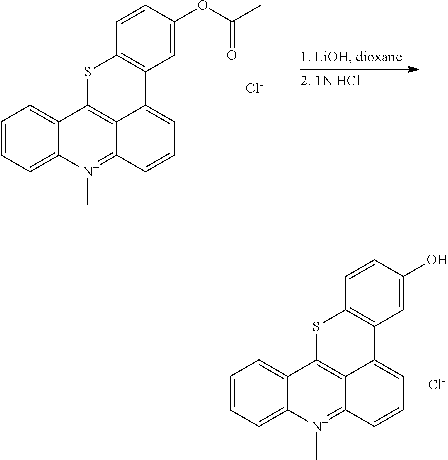 [SMILES: CC(=O)Oc1ccc2c(c1)-c1cccc3c1c(c1ccccc1[n+]3C)S2 ; [Cl-] ; C[n+]1c2ccccc2c2c3c(cccc31)-c1cc(O)ccc1S2 ; [Cl-]]

1N LiOH (250 μL, 0.25 mmol, 4 equ.) was added to a suspension of 3-acetoxy-8-methylthiochromeno[4,3,2-kl]acridin-8-ium chloride (29 mg, 0.0737 mmol; Example 14) in dioxane (2 mL) and the resulting green/blue suspension was stirred for 2 h, after which, LCMS analysis of the reaction mixture showed completion of the reaction. The reaction was quenched with 1N HCl and the resulting purple suspension was filtered. The product was washed successively with water, THF, diethyl ether and dried under suction to give 9 mg of the title compound as a dark purple solid (35%).

### Characterising Data:

δ_{H} (DMSO-d₆): 10.7 (1H, s), 8.60-8.63 (2H, m), 8.45-8.50 (3H, m), 8.24-8.28 (1H, ddd, J=8.8, 6.8, 1.2), 8.07 (1H, d, J=2.3), 7.90-7.92 (1H, d, J=8.7), 7.84-7.87 (1H, ddd, J=8.4, 6.9, 0.7), 7.26-7.29 (1H, dd, J=8.8, 2.4), 4.45 (3H, s).

m/z (ES⁺): 316 (M⁺)

### Example 15

### Preparation of 2-(methoxycarbonyl)-8-methylthiochromeno[4,3,2-kl]acridin-8-ium chloride

### Step 1: Preparation of methyl thiochromeno[4,3,2-kl]acridine-2-carboxylate and methyl thiochromeno[4,3,2-kl]acridine-4-carboxylate

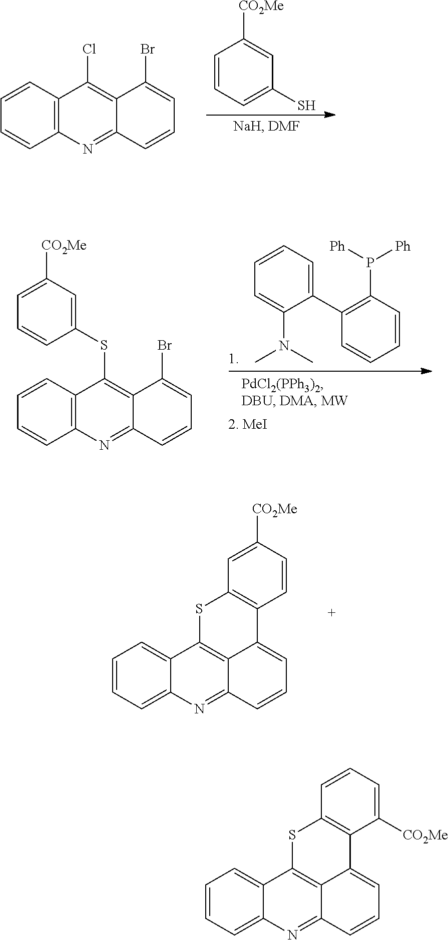

Methyl 3-mercaptobenzoate (474 mg, 2.82 mmol, 1.05 equ.) was added dropwise to a suspension of 1-bromo-9-chloroacridine (785 mg, 2.68 mmol) in anhydrous DMF (8 mL) at 0° C. and the mixture was stirred for 5 min. before the addition of sodium hydride (60% in mineral oil, 113 mg, 2.82 mmol, 1.05 equ.). The mixture was stirred at 0° C. for 30 min., then at room temperature for 1 h. The mixture was poured into ice and the precipitate was filtered, washed with water and dried under suction to give 1.15 g of methyl 3-(1-bromoacridin-9-ylthio)benzoate as a yellow solid. The product was used without purification in the next step.

A mixture of methyl 3-(1-bromoacridin-9-ylthio)benzoate (200 mg, 0.471 mmol), DBU (360 μL, 2.35 mmol, 5 equ.), 2-Diphenylphosphino-2′-(N,N-dimethylamino)biphenyl (18 mg, 0.0471 mmol, 0.1 equ.), and PdCl₂(PPh₃)₂ (16 mg, 0.0236 mmol, 0.05 equ.) in anhydrous DMA (2 mL) was heated at 180° C. under microwave irradiation (300 W, 300 psi) for 5 min. (hold time 5 min.). An excess of methyl iodide was added and the reaction mixture was stirred overnight, then poured into ice. The product was extracted with DCM and the combined organic extracts were washed with water, dried over MgSO₄ and concentrated under vacuum. The residual DMA solution was poured into ice and the precipitate was filtered, washed with water and dried under suction. The resulting solid was dissolved in DCM:MeOH, absorbed on silica and the regioisomers were separated by flash chromatography (gradient elution DCM:MeOH 100%-99.5%-99%-98%) to give 16 mg of pure methyl thiochromeno[4,3,2-kl]acridine-4-carboxylate and 34 mg of a 4:1 mixture of methyl thiochromeno[4,3,2-kl]acridine-2/4-carboxylates as yellow solids (31% combined yield).

### methyl thiochromeno[4,3,2-kl]acridine-4-carboxylate

δ_{H} (CDCl₃): 8.12-8.14 (2H, d, J=8.2), 8.05-8.07 (1H, d, J=8.5), 7.72-7.75 (1H, ddd, J=8.6, 6.7, 1.2), 7.66-7.68 (1H, dd, J=8.6, 7.6), 7.45-7.53 (3H, m), 7.39-7.41 (1H, d, J=7.2), 7.33-7.35 (1H, dd, J=7.7), 3.75 (3H, s).

m/z (ES⁺): 344 (MH⁺)

### Step 2: Preparation of 2-(methoxycarbonyl)-8-methylthiochromeno[4,3,2-kl]acridin-8-ium chloride

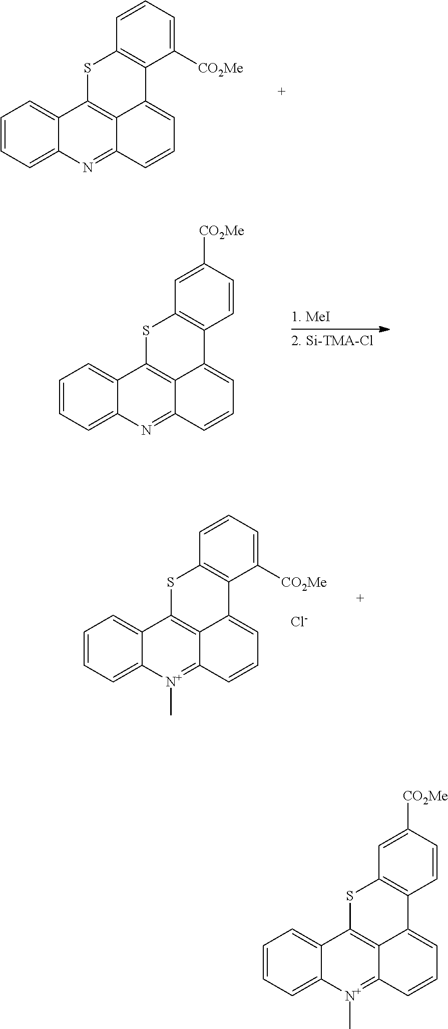 [SMILES: COC(=O)c1cccc2c1-c1cccc3nc4ccccc4c(c13)S2 ; COC(=O)c1ccc2c(c1)Sc1c3ccccc3nc3cccc-2c13 ; COC(=O)c1cccc2c1-c1cccc3c1c(c1ccccc1[n+]3C)S2 ; [Cl-] ; COC(=O)c1ccc2c(c1)Sc1c3ccccc3[n+](C)c3cccc-2c13]

A suspension of a 4:1 mixture of methyl thiochromeno[4,3,2-kl]acridine-2/4-carboxylates (34 mg, 0.099 mmol) in methyl iodide (4 mL) was heated in a sealed tube at 100° C. overnight. The mixture was concentrated under vacuum and the resulting solid was dissolved in DCM:MeOH, absorbed on silica and purified by flash chromatography (gradient elution DCM:MeOH 98%-97%-96.5%-96%-94%-92%-90%) to give 19 mg of the title compound a dark purple solid.

### Characterising Data:

δ_{H} (DMSO-d₆): 8.87-8.97 (1H, d, J=8.6), 8.87-8.89 (1H, d, J=7.2), 8.51-8.63 (5H, m), 8.32-8.35 (1H, m), 8.17-8.19 (1H, dd, J=8.6, 1.2), 7.93-7.97 (1H, dd, J=8.0, 7.5), 4.53 (3H, s), 3.98 (3H, s).

m/z (ES⁺): 358 (M⁺)

### Example 16

### Preparation of 4-(methoxycarbonyl)-8-methylthiochromeno[4,3,2-kl]acridin-8-ium iodide

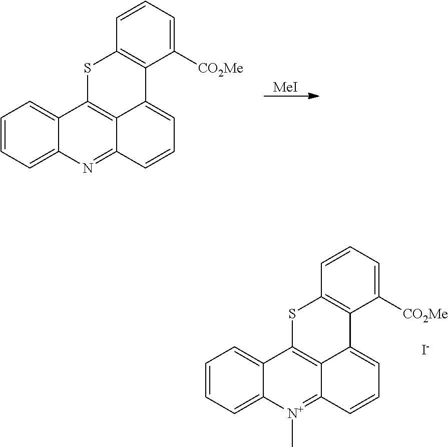 [SMILES: "MeI" ; COC(=O)c1cccc2c1-c1cccc3nc4ccccc4c(c13)S2 ; COC(=O)c1cccc2c1-c1cccc3c1c(c1ccccc1[n+]3C)S2 ; [I-]]

A suspension of methyl thiochromeno[4,3,2-kl]acridine-4-carboxylate (16 mg, 0.0466 mmol) in methyl iodide (3 mL) was heated in a sealed tube at 100° C. overnight, then at 120° C. for 20 h. The mixture was concentrated under vacuum and the resulting solid was dissolved in DCM:MeOH, absorbed on silica and purified by flash chromatography (gradient elution DCM:MeOH 99%-98.5%-98%-96%-94). The product was triturated with diethyl ether, filtered, washed with diethyl ether and dried under suction to give 8 mg of title compound as a dark purple solid (35%).

### Characterising Data:

δ_{H} (DMSO-d₆): 8.67-8.69 (1H, d, J=8.9), 8.60-8.65 (2H, m), 8.49-8.54 (1H, dd, J=8.4, 8.2), 8.34-8.38 (1H, m), 8.25-8.27 (1H, d, J=8.3), 7.84-7.97 (4H, m), 4.57 (3H, s), 3.87 (3H, s).

m/z (ES⁺): 358 (M⁺)

### Example 17

### Preparation of 3-(methoxymethoxy)thiochromeno[4,3,2-kl]acridine

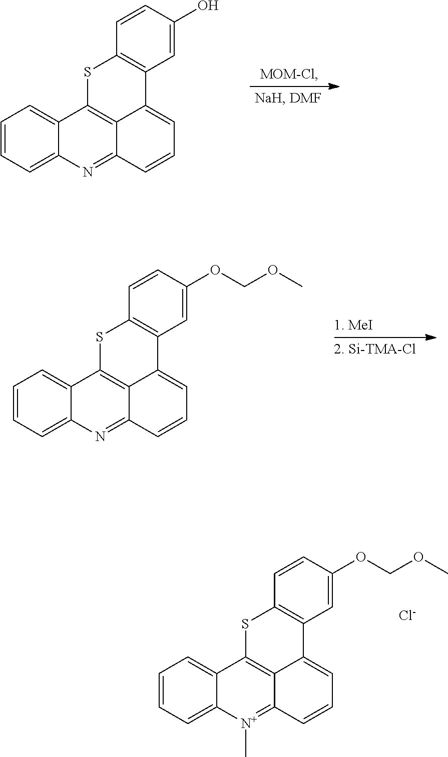 [SMILES: Oc1ccc2c(c1)-c1cccc3nc4ccccc4c(c13)S2 ; COCOc1ccc2c(c1)-c1cccc3nc4ccccc4c(c13)S2 ; COCOc1ccc2c(c1)-c1cccc3c1c(c1ccccc1[n+]3C)S2 ; [Cl-]]

Sodium hydride (60% in mineral oil, 9 mg, 0.234 mmol, 1.1 equ.) was added to a solution of thiochromeno[4,3,2-kl]acridin-3-ol (64 mg, 0.212 mmol) in anhydrous DMF (3 mL) and the mixture was stirred for 5 min. before the addition of MOM chloride (20 μL, 0.234 mmol, 1.1 equ.). The mixture was stirred overnight and was poured into ice. The resulting fine, dark purple suspension was neutralized by the addition of 5N NaOH. The resulting orange/light brown precipitate was filtered, washed with water and dried under suction. The product was dissolved in DCM:MeOH, absorbed on silica and purified by flash chromatography (gradient DCM:MeOH 100%-99%-98%-97%-96%) to give 24 mg of 3-(methoxymethoxy)thiochromeno[4,3,2-kl]acridine as an orange solid. (m/z (ES⁺): 346 (MH⁺)). The product was used without further characterization in the next step.

A suspension of 3-(methoxymethoxy)thiochromeno[4,3,2-kl]acridine (24 mg, 0.0695 mmol) in methyl iodide (3 mL) was heated in a sealed tube at 100° C. overnight. The mixture was cooled to room temperature and was concentrated under vacuum. The resulting solid was triturated with diethyl ether, filtered, washed with diethyl ether and dried under suction. The product was dissolved in DCM:MeOH, silica bound tetramethylammonium chloride (1.18 mmol/g, 180 mg) was added and the mixture was stirred overnight. The mixture was filtered through Celite and the filtrate was concentrated under vacuum. The resulting dark purple solid was triturated in ethyl acetate, filtered, washed with diethyl ether and dried under suction to give 18 mg of the title compound as a dark purple solid.

### Characterising Data:

δ_{H} (DMSO-d₆): 8.79-8.82 (1H, m), 8.62-8.64 (1H, d, J=7.9), 8.45-8.53 (3H, m), 8.33-8.34 (1H, d, J=2.0), 8.26-8.30 (1H, dd, J=8.6, 8.0), 8.02-8.05 (1H, d, J=8.7), 7.86-7.90 (1H, dd, J=8.1, 7.3), 7.49-7.52 (1H, dd, J=8.7, 2.0), 5.48 (2H, s,), 4.49 (3H, s), 3.52 (3H, s).

m/z (ES⁺): 360 (M⁺).

### Example 18

### Preparation of 4-acetamido-8-methylthiochromeno[4,3,2-kl]acridin-8-ium iodide

### Step 1: Preparation of thiochromeno[4,3,2-kl]acridin-4-amine

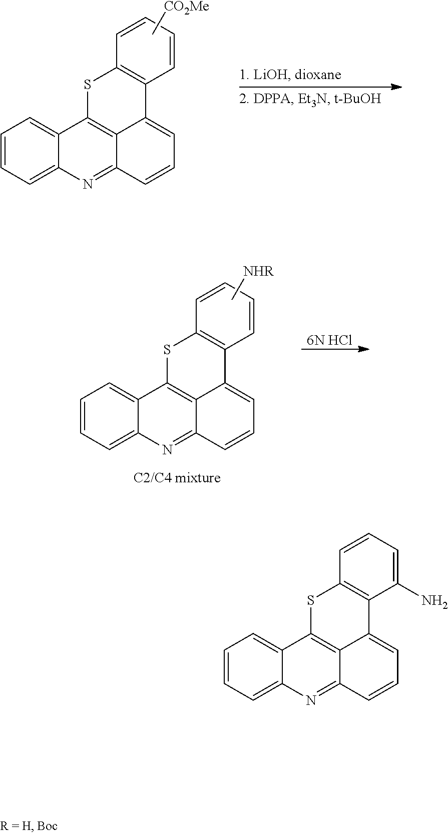

1N LiOH (4 mL, 0.400 mmol, 3.2 equ.) was added to a suspension of methyl thiochromeno[4,3,2-kl]acridine-2-carboxylate (430 mg, 0.125 mmol) in dioxane (20 mL) and the mixture was refluxed overnight. The mixture was concentrated under vacuum and ice was added. The mixture was acidified to pH 4.5 with 1N HCl and the precipitate was filtered, dried under suction and then in a vacuum oven for 3 days to give 382 mg of a C2/C4 mixture of thiochromeno[4,3,2-kl]acridine-carboxylic acids hydrochloride as a red solid. The product was used without further purification in the next step.

DPPA (72 μL, 0.334 mmol, 1.1 equ.) was added to a suspension of crude thiochromeno[4,3,2-kl]acridine-2/4-carboxylic acid hydrochloride (100 mg, 0.304 mmol) and Et₃N (47 μL, 0.246 mmol, 1.1 equ.) in ^{t}BuOH (5 mL) and the mixture was gradually heated to 70° C. for 1 h. A further 2.2 equ. of Et₃N and 2.2 equ. of DPPA were added and the mixture was heated at 70° C. for 1 h and was allowed to cool to r.t. The mixture was concentrated to dryness and the residue was dissolved in DCM. Water was added and the product was extracted with DCM. The combined organic extracts were washed with sat. NaHCO₃, dried over MgSO₄ and concentrated under vacuum.

A suspension of the crude product obtained above in 6N HCl (3 mL) was stirred at r.t for 2 h. The mixture was concentrated to dryness under vacuum and ice was added to the resulting red solid. The mixture was basified with 1N NaOH and the product was extracted with DCM. The combined organic extracts were dried over MgSO₄, then concentrated under vacuum. The product was dissolved in DCM:MeOH and a crude product (20 mg) obtained from a separate experiment was combined. The mixture was absorbed on silica and the product was purified by flash chromatography (gradient elution DCM:MeOH 100%-99%-98%-97.5%-96%) to give 12 mg of the title compound (13% yield).

### Characterising Data:

δ_{H} (DMSO-d₆): 8.40-8.42 (1H, dd, J=7.0, 1.4), 8.11-8.14 (1H, ddd, J=8.6, 1.2, 0.6), 8.01-8.04 (1H, ddd, J=8.8, 1.2, 0.8), 7.86-7.89 (1H, dd, J=8.7, 1.4), 7.81-7.86 (2H, m), 7.58-7.60 (1H, m), 7.11-7.15 (1H, dd, J=8.0, 7.6), 6.84-6.86 (2H, d, J=7.9), 5.76 (2H, s (br)).

### Step 2: Preparation of 4-acetamido-8-methylthiochromeno[4,3,2-kl]acridin-8-ium iodide

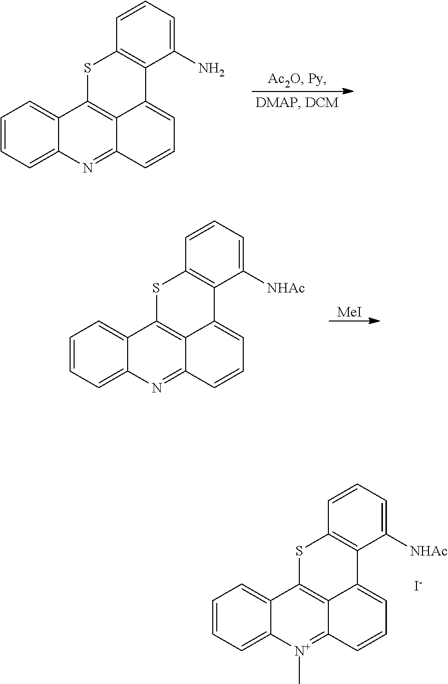 [SMILES: "MeI" ; Nc1cccc2c1-c1cccc3nc4ccccc4c(c13)S2 ; CC(=O)Nc1cccc2c1-c1cccc3nc4ccccc4c(c13)S2 ; CC(=O)Nc1cccc2c1-c1cccc3c1c(c1ccccc1[n+]3C)S2 ; [I-]]

Acetic anhydride (4.7 μL, 0.0499 mmol, 1.5 equ.) was added to a solution of thiochromeno[4,3,2-kl]acridin-4-amine (10 mg, 0.0333 mmol), pyridine (3.7 μL, 0.0499 mmol, 1.5 equ.) and DMAP (catalytic amount) in DCM (1 mL) and the mixture was stirred overnight. 3 equ. of pyridine followed by 3 equ. of acetic anhydride were added and the mixture was stirred for a further 20 h. The addition of reagents was repeated and LCMS analysis of the reaction mixture showed completion of the reaction after a further 20 h.

Water was added and the product was extracted with DCM. The combined organic extracts were washed with sat. NaHCO₃, dried over MgSO₄ and concentrated under vacuum. The residue was dissolved in DCM:MeOH, absorbed on silica and purified by flash chromatography (gradient elution DCM:MeOH 100%-98%-97%-96%-95%) to give 5 mg of the title compound as a yellow/orange solid (44%). (m/z (ES⁺): 343 (MH⁺)). The product was used without further characterization in the next step.

A solution of N-(thiochromeno[4,3,2-kl]acridin-4-yl)acetamide (5 mg, 0.0146 mmol) in methyl iodide (3 mL) was heated at 120° C. in a sealed tube overnight. The mixture was cooled to room temperature and concentrated to dryness under vacuum. The resulting solid was triturated in diethyl ether, then filtered, washed with diethyl ether and dried under suction to give 3 mg of the title compound as a dark purple solid (43%).

### Characterising Data:

δ_{H} (DMSO-d₆): 10.4 (1H, s (br)), 8.73-8.75 (1H, d, J=7.4), 8.66-8.68 (1H, dd, J=8.5, 0.9), 8.57-8.59 (1H, d, J=8.8), 8.51-8.53 (1H, d, J=8.8), 8.45-8.47 (1H, dd, J=9.0, 7.6), 8.31-8.35 (1H, ddd, J=9.0, 6.8, 1.0), 7.91-7.95 (2H, m), 7.71-7.75 (1H, dd, J=7.9), 7.61-7.62 (1H, d, J=7.8), 4.55 (3H, s), 2.07 (3H, s).

m/z (ES⁺): 357 (M⁺).

Note: The structure of the product was confirmed by COSY and NOESY analyses.

### Example 19

### Preparation of 5,10-dimethoxy-8-methylthiochromeno[4,3,2-kl]acridin-8-ium iodide

### Step 1: Preparation of methyl 2-(3-bromo-4-methoxyphenylamino)-4-methoxybenzoate

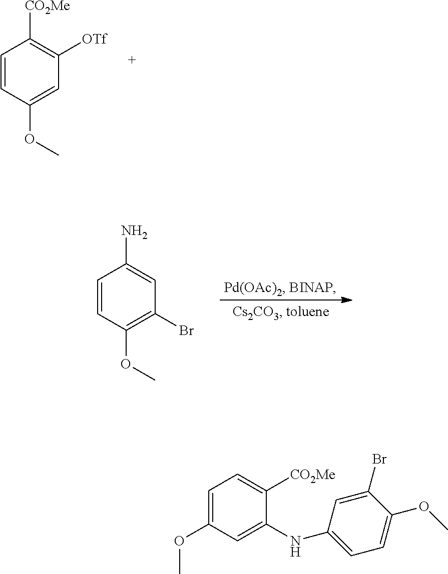

Nitrogen was bubbled through a suspension of 3-bromo-4-methoxyaniline (2.5 g, 12.4 mmol, 1.2 equ.), methyl 4-methoxy-2-(trifluoromethylsulfonyloxy)benzoate (3.24 g, 10.3 mmol) and cesium carbonate (4.37 g, 13.4 mmol, 1.3 equ.) in anhydrous toluene (110 mL) for 10 minutes before the addition of BINAP (0.64 g, 1.03 mmol, 0.1 equ.) and palladium acetate (116 mg, 0.516 mmol, 0.05 equ.). The reaction mixture was refluxed overnight, then cooled to room temperature and concentrated to dryness under vacuum. The resulting dark residue was absorbed on silica and the product was purified by flash chromatography (gradient elution Hexane:EtOAc 20:1-10:1 to give 2.4 g of the title compound as an off-white solid (64%).

### Characterising Data:

m/z (ES⁺): 368 (MH⁺, ⁸¹Br), 366 (MH⁺, ⁷⁹Br)

The product was used without further characterization in the next step.

### Step 2: preparation of 1-bromo-9-chloro-2,6-dimethoxyacridine

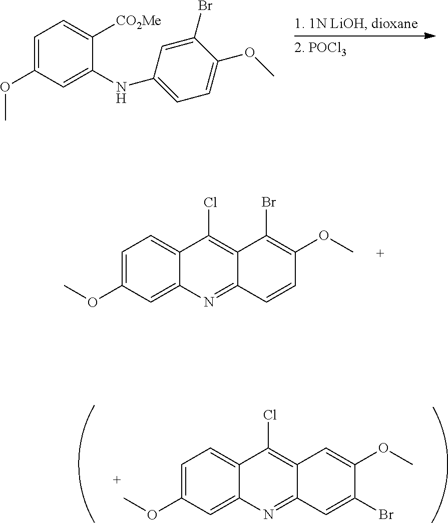

1N LiOH (10 mL, 10 mmol, 1.5 equ.) was added to a solution of methyl 2-(3-bromo-4-methoxyphenylamino)-4-methoxybenzoate (2.4 g, 6.55 mmol) in dioxane (45 mL) and the resulting hazy solution was stirred at 50° C. overnight, after which LCMS analysis of the reaction mixture showed 80% conversion. 10 mL of 1N LiOH were added and the reaction mixture was heated at 50° C. for 5 h, then at room temperature overnight. The mixture was concentrated to dryness under vacuum and the resulting white residue was suspended in 3M HCl (50 mL). The precipitate was filtered, washed successively with water and hexane to give 2.07 g of 2-(3-bromo-4-methoxyphenylamino)-4-methoxybenzoic acid as a white solid (90%). m/z (ES⁺): 354 (MH⁺, ⁸¹Br), 352 (MH⁺, ⁷⁹Br). The product, which was 90% pure as estimated by LCMS analysis, was used without further purification and characterization in the next step.

A solution of 2-(3-bromo-4-methoxyphenylamino)-4-methoxybenzoic acid (2.07 g, 5.90 mmol) in phosphorus oxychloride (10 mL) was refluxed for 2 hours and was then allowed to cool to room temperature. The reaction mixture was concentrated under vacuum and the resulting black residue was poured in a stirred mixture of ice (20 g), chloroform (8 mL) and ammonia (8 mL). The mixture was stirred for 20 minutes and the organic layer was separated. The aqueous layer was extracted with chloroform, and the combined organic were washed with brine, dried over MgSO₄ and concentrated under vacuum. The resulting yellow solid was dissolved in DCM, absorbed on silica and the two regioisomers were separated by flash chromatography (gradient elution hexane:EtOAc 10:1-5:1-1:1-1:2-1:3-1:4) to give 893 mg of 3-bromo-9-chloro-2,6-dimethoxyacridine and 630 mg of the title compound as a yellow solid. (74% combined yield)

### Characterising Data:

δ_{H} (DMSO-d₆): 8.37-8.40 (1H, dd, J=8.9, 1.0), 8.19-8.20 (1H, d, J=9.5), 7.94-7.97 (1H, d, J=9.7), 7.44-7.47 (2H, m), 4.07 (3H, s), 3.99 (3H, s).

### Step 3: Preparation of 5,10-dimethoxy-8-methylthiochromeno[4,3,2-kl]acridin-8-ium iodide

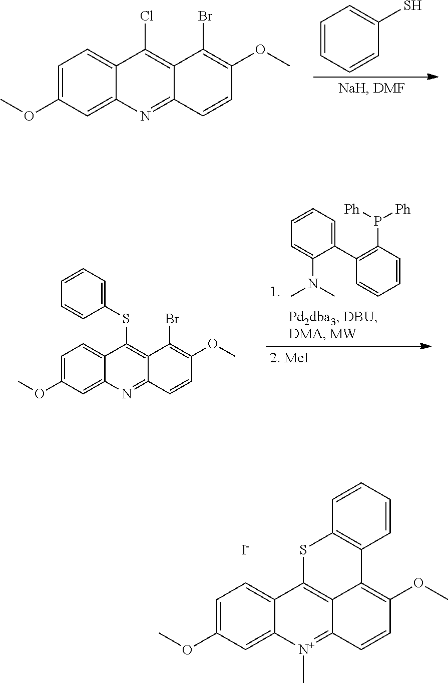

Thiophenol (230 μL, 2.25 mmol, 1.5 equ.) was added to a suspension of 1-bromo-9-chloro-2,6-dimethoxyacridine (530 mg, 1.5 mmol) in anhydrous DMF (8 mL) at 0° C. and the resulting red suspension was stirred for 5 minutes before the addition of sodium hydride (60% in mineral oil, 90 mg, 2.25 mmol, 1.5 equ.) The resulting yellow suspension was stirred overnight and the precipitate was filtered, then washed twice with hexane to give 360 mg of 1-bromo-2,6-dimethoxy-9-(phenylthio)acridine as a yellow solid. The filtrate was poured into ice (50 g), and the suspension was filtered. The solid was triturated in hexane with the minimum amount of DCM to aid trituration and the solid was filtered, then dried in a vacuum oven overnight, to give a second crop of the product (160 mg) as a yellow solid (81% combined yield). m/z (ES⁺): 428 (MH⁺, ⁸¹Br), 426 (MH⁺, ⁷⁹Br). The product was used crude without further characterization in the next step.

A mixture of 1-bromo-2,6-dimethoxy-9-(phenylthio)acridine (50 mg, 0.117 mmol), DBU (20 μL, 0.134 mmol, 1.15 equ.), 2-Diphenylphosphino-2′-(N,N-dimethylamino)biphenyl (5 mg, 0.0131 mmol, 0.1 equ.), and Pd₂dba₃ (5 mg, 0.0055 mmol, 0.05 equ.) in DMA (1 mL) was heated at 180° C. under microwave irradiation (300 W, 300 psi) for 8 min. (hold time 8 min., without cooling). A total of three runs were carried out and the combined reaction mixtures were poured into ice. The resulting precipitate was filtered and dried under suction overnight. The product was dissolved in DCM:MeOH, absorbed on silica and purified by flash chromatography (gradient elution DCM:MeOH 100%-99.9%-99.7%-99.6%-99.5%-99%) to give 27 mg of 5,10-dimethoxythiochromeno[4,3,2-kl]acridine in 74% purity (LCMS analysis, m/z (ES⁺): 346 (MH⁺)). The product was used without further purification in the next step.

A mixture of 5,10-dimethoxythiochromeno[4,3,2-kl]acridine (27 mg, 0.0782 mmol) in methyl iodide (2 mL) was stirred at 65° C. for 2 hours in sealed tube, then at room temperature overnight. The reaction mixture was concentrated under vacuum and the resulting solid was dissolved in DCM:MeOH and absorbed into silica. The product was purified by flash chromatography (gradient elution DCM: MeOH, 100%-99%-98.5%-98%-97%-95%-83%) to give the title compound in 50% yield.

### Characterising Data:

δ_{H} (MeOD): 9.02-9.04 (1H, m), 8.50-8.53 (1H, d, J=9.4), 8.40-8.42 (1H, d, J=9.8), 8.31-8.33 (1H, d, J=9.9), 7.74-7.78 (1H, m), 7.54-7.57 (2H, m), 7.46-7.47 (1H, d, J=2.2), 7.40-7.44 (1H, dd, J=9.4, 2.2), 4.45 (3H, s), 4.18 (3H, s).

### Example 20

### Preparation of 3-(methoxycarbonyl)-8-methylthiochromeno[4,3,2-kl]acridin-8-ium iodide and chloride

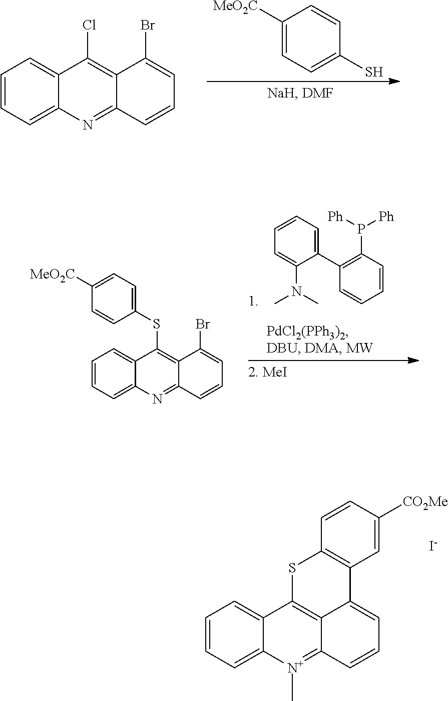 [SMILES: Clc1c2ccccc2nc2cccc(Br)c12 ; COC(=O)c1ccc(S)cc1 ; COC(=O)c1ccc(Sc2c3ccccc3nc3cccc(Br)c23)cc1 ; CN(C)c1ccccc1-c1ccccc1P(c1ccccc1)c1ccccc1 ; COC(=O)c1ccc2c(c1)-c1cccc3c1c(c1ccccc1[n+]3C)S2 ; [I-]]

To a solution of 9-chloro-1-bromoacridine (1.355 g, 4.631 mmol) in anhydrous DMF (60 mL) under nitrogen was added methyl 4-mercaptobenzoate (0.779 g, 4.631 mmol, 1 equ.) and the suspension was stirred at room temperature. Sodium hydride (60% in mineral oil, 0.222 g, 15.557 mmol, 1.2 equ.) was added in one portion and the resulting suspension was stirred for 30 min. before the addition of water (50 mL). The resulting solid was collected by filtration, dried under suction, then in a vacuum oven at 40° C. overnight to give 1.95 g of methyl 4-(1-bromoacridin-9-ylthio)benzoate as a yellow solid. (99%). The product was used without further purification in the next step.

A solution of methyl 4-(1-bromoacridin-9-ylthio)benzoate (50 mg, 0.188 mmol), PdCl₂(PPh₃)₂ (catalytic amount), 1.0 mL of a 4.5 mg/mL DMA stock solution of 2-diphenylphosphino-2-(N,N-dimethylamino)biphenyl ligand (0.019 mmol, 0.1 equ.), DBU (88 μL, 0.589 mmol, 5 equ.) in anhydrous DMA (4 mL) was heated under microwave radiations (300 W, 300 psi, 180° C.) for 5 min. 9 consecutive runs were carried out and the combined reaction mixtures were concentrated under vacuum. The residue was dissolved in DCM:MeOH, absorbed on silica and purified by two successive flash chromatographies (gradient elution DCM:MeOH 100% to 85% (1% increments)) to give 110 mg of methyl thiochromeno[4,3,2-kl]acridine-3-carboxylate. (m/z (ES⁺): 344 (MH⁺). The product was used without further characterization in the next step.

A solution of methyl thiochromeno[4,3,2-kl]acridine-3-carboxylate (110 mg, 0.320 mmol) in methyliodide (5 mL) was heated at 75° C. in a sealed tube overnight and the mixture was allowed to cool to room temperature. The mixture was concentrated to dryness under vacuum and the product was dissolved in DCM:MeOH, absorbed on silica and purified by flash chromatography (gradient elution DCM:MeOH 100% to 85% by 1% increment) to give 34 mg of 3-(methoxycarbonyl)-8-methylthiochromeno[4,3,2-kl]acridin-8-ium iodide.

The solid was dissolved in DCM and 178 mg of silica bound tetramethylammonium chloride (1.18 mmol/g). The mixture was stirred overnight, then loaded on a column packed with silica. Elution with 5% DCM:MeOH gave 19 mg of 3-(methoxycarbonyl)-8-methylthiochromeno[4,3,2-kl]acridin-8-ium chloride.

### Characterising Data:

δ_{H} (MeOD): 9.10-9.11 (1H, d, J=1.6), 8.72-8.75 (1H, dd, J=4.4), 8.65-8.48 (1H, dd, J=8.6, 1.2), 8.44-8.48 (3H, m), 8.26-8.31 (1H, ddd, J=9.0, 6.8, 1.3), 8.15-8.17 (1H, dd, J=8.4, 1.6), 7.96-7.98 (1H, d, J=8.3), 7.85-7.89 (1H, ddd, J=8.6, 0.8, 0.9), 4.55 (3H, s), 4.02 (3H, s).

m/z (ES⁺): 358 (M⁺)

1. A compound of formula I: 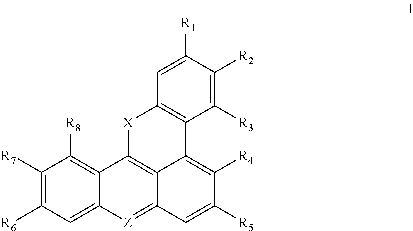
wherein:
X is O, S, SO or SO₂;
Z is N or N⁺-Q, wherein Q is selected from the group consisting of O⁻, (1-6C)alkyl, (2-6C)alkenyl and (2-6C)alkynyl, or Q is a group of the formula:
-L¹-Q¹
wherein:
L¹ is (1-6C)alkylene, (2-6C)alkenylene or (2-6C)alkynylene, each of which is optionally substituted with one or more (1-4C)alkyl groups;
Q¹ is selected from the group consisting of —OR₉, —NR₉R₁₀, —S(O)ₚR₉ (wherein p is 0, 1 or 2), —C(O)R₉, —C(O)OR₉, —OC(O)R₉, —C(O)NR₉R₁₀, —N(R₁₀)C(O)R₉, —N(R₁₀)CON(R₁₀)R₉—, SO₂N(R₉)—, —N(R₉)SO₂—, (3-8C)cycloalkyl, aryl, heterocyclyl, and heteroaryl, and wherein the (3-8C)cycloalkyl, aryl, heterocyclyl, or heteroaryl group is optionally substituted by one or more substituents independently selected from the group consisting of halogen, cyano, nitro, hydroxy, amino and (1-4C)alkoxy;
R₉ is selected from the group consisting of hydrogen, (1-6C)alkyl, (3-8C)cycloalkyl, aryl, heterocyclyl, and heteroaryl, and wherein the (1-6C)alkyl, (3-8C)cycloalkyl, aryl, heterocyclyl, or heteroaryl group is optionally substituted by one or more substituents independently selected from the group consisting of halogen, cyano, nitro, hydroxy, amino and (1-4C)alkoxy;
R₁₀ is selected from hydrogen or (1-6C)alkyl;
R₁, R₂, R₃, R₄, R₅, R₆, R₇ and R₈ are each independently selected from the group consisting of hydrogen, halogen, trifluoromethyl, cyano, nitro, hydroxy, mercapto, amino, formyl, carboxy, carbamoyl, ureido, (1-6C)alkyl, (2-6C)alkenyl, (2-6C)alkynyl,
and a group of the formula:
-L²-L³-R₂₀
wherein
L² is absent or a linker group of the formula —[CR₁₁R₁₂]ₙ— in which n is an integer selected from 1, 2, 3 or 4 and R₁₁ and wherein R₁₂ are each independently selected from hydrogen or (1-4C)alkyl;
L³ is absent or is selected from the group consisting of O, S, SO, SO₂, N(R₁₃), C(O), CH(OR₁₃), C(O)O, OC(O), C(O)N(R₁₃), N(R₁₃)C(O), N(R₁₃)C(O)N(R₁₄), S(O)₂N(R₁₃), and N(R₁₃)SO₂, wherein R₁₃ and R₁₄ are each independently selected from hydrogen or (1-4C)alkyl; and
R₂₀ is selected from the group consisting of (1-6C)alkyl, aryl, aryl-(1-6C)alkyl, (3-6C)cycloalkyl, (3-6C)cycloalkyl-(1-6C)alkyl, (3-6C)cycloalkenyl, (3-6C)cycloalkenyl-(1-6C)alkyl, heteroaryl, heteroaryl-(1-6C)alkyl, heterocyclyl and heterocyclyl-(1-6C)alkyl, and wherein R₂₀ is optionally further substituted by one or more substituents independently selected from the group consisting of halogen, cyano, nitro, hydroxy, amino and (1-4C)alkoxy;
or a pharmaceutically acceptable salt, hydrate or solvate thereof.

2. A compound according to claim 1, wherein X is O.

3. A compound according to claim 1, wherein X is S.

4. A compound according to claim 1, wherein Z is N.

5. A compound according to claim 1, wherein Z is N⁺-Q.

6. A compound according to claim 5, wherein Q is selected from the group consisting of O⁻, (1-6C)alkyl, (2-6C)alkenyl and (2-6C)alkynyl, or Q is a group of the formula:
-L¹-Q¹
wherein:
L¹ is (1-6C)alkylene which is optionally substituted with one or more (1-4C)alkyl groups;
Q¹ is selected from the group consisting of —OR₉, —NR₉R₁₀, —S(O)ₚR₉ (wherein p is 0, 1 or 2), —C(O)R₉, —C(O)OR₉, —OC(O)R₉, —C(O)NR₉R₁₀, —N(R₁₀)C(O)R₉, —N(R₁₀)CON(R₁₀)R₉—, —SO₂N(R₉)—, —N(R₉)SO₂—, (3-8C)cycloalkyl, aryl, heterocyclyl, and heteroaryl, and wherein the (3-8C)cycloalkyl, aryl, heterocyclyl or heteroaryl group is optionally substituted by one or more substituents independently selected from the group consisting of halogen, cyano, nitro, hydroxy, amino and (1-4C)alkoxy;
R₉ is selected from the group consisting of hydrogen, (1-6C)alkyl, (3-8C)cycloalkyl, aryl, heterocyclyl, and heteroaryl, and wherein the (1-6C)alkyl, (3-8C)cycloalkyl, aryl, heterocyclyl, or heteroaryl group is optionally substituted by one or more substituents independently selected from the group consisting of halogen, cyano, nitro, hydroxy, amino and (1-4C)alkoxy; and
R₁₀ is selected from hydrogen or (1-6C)alkyl.

7. A compound according to claim 5, wherein Q is selected from O⁻ or (1-6C)alkyl or Q is a group of the formula:
-L¹-Q¹
wherein:
L¹ is (1-2C)alkylene;
Q¹ is selected from the group consisting of —OR₉, —NR₉R₁₀, —S(O)ₚR₉ (wherein p is 0, 1 or 2), —C(O)R₉, —C(O)OR₉, —OC(O)R₉, —C(O)NR₉R₁₀, —N(R₁₀)C(O)R₉, —N(R₁₀)CON(R₁₀)R₉—, —SO₂N(R₉)—, —N(R₉)SO₂—, (3-6C)cycloalkyl, aryl, heterocyclyl, and heteroaryl, and wherein the (3-6C)cycloalkyl, aryl, heterocyclyl or heteroaryl group is optionally substituted by one or more substituents independently selected from the group consisting of halogen, cyano, nitro, hydroxy, amino and (1-4C)alkoxy;
R₉ is selected from hydrogen or (1-4C)alkyl; and
R₁₀ is selected from hydrogen of (1-2C)alkyl.

8. A compound according to claim 1, wherein R₁, R₂, R₃, R₄, R₅, R₆, R₇ and R₈ are each independently selected from the group consisting of hydrogen, halogen, trifluoromethyl, cyano, nitro, hydroxy, mercapto, amino, formyl, carboxy, carbamoyl, ureido, (1-6C)alkyl, (2-6C)alkenyl, (2-6C)alkynyl, and a group of the formula:
-L²-L³-R₂₀
wherein
L² is absent or a linker group of the formula —[CR₁₁R₁₂]ₙ— in which n is an integer selected from 1 or 2, and R₁₁ and R₁₂ are each independently selected from hydrogen or (1-2C)alkyl;
L³ is absent or is selected from the group consisting of O, S, SO, SO₂, N(R₁₃), C(O), CH(OR₁₃), C(O)O, OC(O), C(O)N(R₁₃), N(R₁₃)C(O), N(R₁₃)C(O)N(R₁₄), S(O)₂N(R₁₃), and N(R₁₃)SO₂, wherein R₁₃ and R₁₄ are each independently selected from hydrogen or (1-2C)alkyl; and
R₂₀ is (1-6C)alkyl, aryl, aryl-(1-6C)alkyl, (3-6C)cycloalkyl, (3-6C)cycloalkyl-(1-6C)alkyl, heteroaryl, heteroaryl-(1-6C)alkyl, heterocyclyl and heterocyclyl-(1-6C)alkyl, and wherein R₂₀ is optionally further substituted by one or more substituents independently selected from the group consisting of halogen, cyano, nitro, hydroxy, amino and (1-4C)alkoxy.

9. A compound according to claim 1, wherein R₁, R₂, R₃, R₄, R₅, R₆, R₇ and R₈ are each independently selected from the group consisting of hydrogen, halogen, trifluoromethyl, cyano, nitro, hydroxy, mercapto, amino, carbamoyl, (1-6C)alkyl, and a group of the formula:
-L²-L³-R₂₀
wherein
L² is absent or a methylene linker;
L³ is absent or is selected from the group consisting of O, S, SO, SO₂, N(R₁₃), C(O), CH(OR₁₃), C(O)O, OC(O), C(O)N(R₁₃), N(R₁₃)C(O), N(R₁₃)C(O)N(R₁₄), S(O)₂N(R₁₃), and N(R₁₃)SO₂, wherein R₁₃ and R₁₄ are each independently selected from hydrogen or (1-2C)alkyl; and
R₂₀ is (1-6C)alkyl or (3-6C)cycloalkyl, and wherein R₂₀ is optionally further substituted by one or more substituents independently selected from the group consisting of halogen, cyano, nitro, hydroxy, amino and (1-2C)alkoxy.

10. A compound according to claim 1, wherein up to four of R₁, R₂, R₃, R₄, R₅, R₆, R₇ and R₈ are a substituent group other than hydrogen.

11. A compound according to claim 10, wherein one or two of R₁, R₂, R₃, R₄, R₅, R₆, R₇ and R₈ are a substituent group other than hydrogen.

12. A compound of structural formula II shown below: 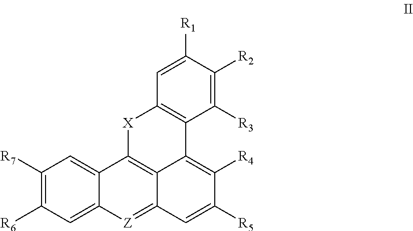
wherein X, Z, R₁, R₂, R₃, R₄, R₅, R₆ and R₇ are as defined in claim 1, or a pharmaceutically acceptable salt, hydrate or solvate thereof.

13. A compound of claim 1, which is selected from any one of the following:
chromeno[4,3,2-kl]acridine;
thiochromeno[4,3,2-kl]acridine;
chromeno[4,3,2-kl]acridine 8-oxide;
N-(chromeno[4,3,2-kl]acridin-3-yl)acetamide;
8-methylchromeno[4,3,2-kl]acridin-8-ium;
8-methylthiochromeno[4,3,2-kl]acridin-8-ium;
8-(2-ethoxy-2-oxoethyl)chromeno[4,3,2-kl]acridin-8-ium;
acetamido-8-methylchromeno[4,3,2-kl]acridin-8-ium;
3-methoxy-8-methylthiochromeno[4,3,2-kl]acridin-8-ium;
4-methoxy-8-methylthiochromeno[4,3,2-kl]acridin-8-ium;
3,10-dimethoxy-8-methylthiochromeno[4,3,2-kl]acridin-8-ium;
10-methoxy-2-(methoxycarbonyl)-8-methylthiochromeno[4,3,2-kl]acridin-8-ium;
10-methoxy-4-(methoxycarbonyl)-8-methylthiochromeno[4,3,2-kl]acridin-8-ium;
3-acetoxy-8-methylthiochromeno[4,3,2-kl]acridin-8-ium;
3-hydroxy-8-methylthiochromeno[4,3,2-kl]acridin-8-ium;
2-(methoxycarbonyl)-8-methylthiochromeno[4,3,2-kl]acridin-8-ium;
4-(methoxycarbonyl)-8-methylthiochromeno[4,3,2-kl]acridin-8-ium;
3-(methoxymethoxy)thiochromeno[4,3,2-kl]acridine;
4-acetamido-8-methylthiochromeno[4,3,2-kl]acridin-8-ium;
5,10-dimethoxy-8-methylthiochromeno[4,3,2-kl]acridin-8-ium; and
3-(methoxycarbonyl)-8-methylthiochromeno[4,3,2-kl]acridin-8-ium;
or a pharmaceutically acceptable salt, hydrate or solvate thereof.

14. A pharmaceutical composition which comprises a compound according to claim 1, or a pharmaceutically acceptable, hydrate or solvate thereof, in association with a pharmaceutically-acceptable diluent or carrier.

15-17. (canceled)

18. A method of treating a proliferative disorder comprising administering to a human or animal in need of such treatment a therapeutically effective amount of a compound according to claim 1, or a pharmaceutically acceptable, hydrate or solvate thereof.

19. A method of inhibiting telomerase activity in a cell, the method comprising administering to said cell a compound according to claim 1, or a pharmaceutically acceptable, hydrate or solvate thereof.

20. The method of claim 18, wherein the proliferative disorder is cancer.
